(19) United States
(12) Patent Application Publication
(10) Pub. No.: US 2006/0068472 A1
(43) Pub. Date: Mar. 30, 2006

(54) Compositions to ameliorate protein misfolding and aggregation

(76) Inventors: Guy A. Caldwell, Tuscaloosa, AL (US); Kim A. Caldwell, Tuscaloosa, AL (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/282,495

(22) Filed: Nov. 18, 2005

Related U.S. Application Data
(62) Division of application No. 10/177,104, filed on Jun. 24, 2002.

(51) Int. Cl. C07K 14/705; C12Q 1/68; A01K 67/033; C07H 21/04

(52) U.S. Cl. 435/69.1; 435/320.1; 435/325; 530/350; 435/6; 536/23.5; 800/8

(57) ABSTRACT

The invention relates to polynucleotides comprising polynucleotide sequences corresponding to the tor-1, tor-2, ooc-5, DYT1, and DYT2 genes and parts thereof that encode polypeptide sequences and parts thereof possessing varying degrees of torsin activity, and methods of screening and amplifying polynucleotides encoding polypeptide sequences which encode polypeptides having varying degrees of TOR-1, TOR2, OOC-5 TOR-A, and TOR-B activity. Further, the invention relates to methods of reducing protein aggregation, methods of treating diseases that are caused by protein aggregation, methods of screening potential protein-aggregation-reducing products, methods of screening potential therapeutics of diseases caused by protein aggregation, and pharmaceuticals, therapeutics, and kits comprising polynucleotide sequences corresponding to the tor-1, tor-2, ooc-5, DYT1, and DYT2 genes and/or polypeptides having torsin activity.

### BACKGROUND OF INVENTION

1. Field of the Invention

The invention relates to polynucleotides comprising polynucleotide sequences corresponding to the tor-1, tor-2, ooc-5, DYT1, and DYT2 genes and parts thereof that encode polypeptide sequences and parts thereof possessing varying degrees of torsin activity, and methods of screening and amplifying polynucleotides encoding polypeptide sequences which encode polypeptides having varying degrees of TOR-1, TOR2, OOC-5 TOR-A, and TOR-B activity. Further, the invention relates to methods of reducing protein aggregation, methods of treating diseases that are caused by protein aggregation, methods of screening potential protein-aggregation-reducing products, methods of screening potential therapeutics of diseases caused by protein aggregation, and pharmaceuticals, therapeutics, and kits comprising polynucleotide sequences corresponding to the tor-1, tor-2, ooc-5, DYT1, and DYT2 genes and/or polypeptides having torsin activity.

2. Discussion of the Background

Neuronal damage may be caused by toxic, aggregation-prone proteins. Further, an enormous scope of neurodegenerative disorders is characterized by such neuronal damage. Therefore, these neurodegenerative disorders are inevitably a result of protein aggregation. Genes have been identified that code for such toxic, aggregation-prone proteins which cause these disorders. Further, mutations in such genes result in abnormal processing and accumulation of misfolded proteins. These misfolded proteins are known to result in neuronal damage such as neuronal inclusions and plaques. Therefore, the understanding of the cellular mechanisms and the identification of the molecular tools required for the reduction, inhibition, and amelioration of such misfolded proteins is critical. Further, an understanding of the effects of protein aggregation on neuronal survival will allow the development of rational, effective treatment for these disorders.

Neuronal disorders, including early-onset torsion dystonia are characterized by uncontrolled muscular spasms. Dystonia is set apart in that the muscle spasms are repetitive and rhythmic (Bressman, S B. 1998. Dystonia Current Opinion in Neurology. 11:363-372). The symptoms can range in severity from a writer's cramp to being wheelchair bound. Early-onset torsion dystonia, also called primary dystonia, is distinguished by strong familial ties and the absence of any neural degeneration, which is seen in the other movement disorders. This is most severe form of the disease and is dominantly inherited with a low penetrance (30%-40%) (L. J. Ozelius, et al., Genomics 62, 377 (1999); L. J. Ozelius, et al., Nature Genetics 17, 40 (1997)). Therefore, dystonia is difficult to diagnose and pathologically define. Dystonia affects more than 300,000 people in North America and is more common than Huntington's disease and muscular dystrophy. Treatment is very limited because the disease is poorly understood and options include surgery or injection of botulism toxin to control the muscle contractions.

The molecular basis for torsion dystonia remains unclear. Ozelius et al. identified the causative gene, named TOR1A (DYT1), and mapped it to human chromosome 9q34 (L. J. Ozelius, et al., Nature Genetics 17, 40 (1997)). The TOR1A gene produces a protein named TOR-A. The majority of patients with early onset torsion dystonia have a unique deletion of one codon, which results in a loss of glutamic acid (GAG) residue at the carboxy terminal of TOR-A. A misfunctional torsin protein is produced. Notably, this was the only change observed on the disease chromosome (L. J. Ozlius, et al., Genomics 62, 377 (1999); L. J. Ozelius, et al., Nature Genetics 17, 40 (1997)). A recent paper described an additional deletion of 18 base pairs or 6 amino acids at the carboxy terminus. This is the first mutation identified beyond the GAG deletion (L. J. Ozelius, et al., Nature Genetics 17, 40 (1997)).

In the original paper identifying the TOR1A gene, a nematode torsin-like protein was described, which has since been shown to encode the ooc-5 gene (L. J. Ozelius, et al., Nature Genetics 17, 40 (1997); S. E. Basham, and L. E. Rose, Dev Biol 215 253 (1999)). The TOR-A protein shares a distant similarity (25%-30%) to the AAA+/Hsp 100/Clp family of proteins (chromosome (L. J. Ozelius, et al., Genomics 62, 377 (1999); Neuwald A F, Aravind L, Spouge J L, Koonin E V. 1999. AAA+: A class of chaperone-like ATPases associated with the assembly, operation, and disassembly of protein complexes. Genome Res 9: 27-43). Their tasks are as diverse as their similarities. For example, they perform chaperone functions, regulate protein signaling, and allow for the correct localization of the proteins. However, until the time of the present invention, the function of torsin proteins has not been elucidated and their activities are unknown.

### SUMMARY OF THE INVENTION

The present invention relates to dystonia, dystonia genes, encoded proteins and mutations in dystonia genes that result in a dystonia disorder. In particular, the invention provides isolated nucleic acid molecules coding for torsin proteins, preferably, TOR-2.

The invention further provides purified polypeptides comprising amino acid sequences contained in torsin proteins.

The invention also provides nucleic acid probes for the specific detection of the presence of and mutations in nucleic acids encoding torsin proteins or polypeptides in a sample.

The invention further provides a method of detecting the presence of mutations in nucleic acid encoding a torsin protein in a sample.

The invention also provides a kit for detecting the presence of mutations in a nucleic acid encoding a torsin protein in a sample.

The invention further provides a recombinant nucleic acid molecule comprising, 5′ to 3′, a promoter effective to initiate transcription in a host cell and the above-described isolated nucleic acid molecule.

The invention also provides a recombinant nucleic acid molecule comprising a vector and the above-described isolated nucleic acid molecule.

The invention further provides a method of screening for a compound that reduces, inhibits, ameliorates, or prevents protein aggregation by comparing the amount of protein aggregation in the presence of the compound to the amount of protein aggregation in the absence of the compound. This method of screening is performed in the presence of at least one torsin protein. The torsin protein may be mutated.

The invention further provides a recombinant nucleic acid molecule comprising a sequence complimentary to an RNA sequence encoding an amino acid sequence corresponding to the above-described polypeptide.

The invention also provides a cell that contains the above-described recombinant nucleic acid molecule.

The invention further provides a non-human organism that contains the above-described recombinant nucleic acid molecule.

The invention also provides an antibody having binding affinity specifically to a torsin protein or polypeptide.

The invention further provides a method of detecting a torsin protein or polypeptide in an sample.

The invention also provides a method of measuring the amount of a torsin protein or polypeptide in a sample.

The invention further provides a method of detecting antibodies having binding affinity specifically to a torsin protein or polypeptide.

The invention further provides a diagnostic kit comprising a first container means containing a conjugate comprising a binding partner of the monoclonal antibody and a label.

The invention also provides a hybridoma which produces the above-described monoclonal antibody.

The invention further provides diagnostic methods for dystonia disorders in humans, in particular, torsion dystonia. Preferably, a method of diagnosing the presence or absence of dystonia; predicting the likelihood of developing or a predisposition to develop dystonia in a human is provided herein. The dystonia disorder can be, for example, torsion dystonia. A biological sample obtained from a human can be used in the diagnostic methods. The biological sample can be a bodily fluid sample such as blood, saliva, semen, vaginal secretion, cerebrospinal and amniotic bodily fluid sample. Alternatively or additionally, the biological sample is a tissue sample such as a chorionic villus, neuronal, epithelial, muscular and connective tissue sample. In both bodily fluid and tissue samples, nucleic acids are present in the samples.

The dystonia gene can be the tor-1, tor-2, ooc-5, DYT1, and DYT2 genes, and parts thereof (SEQ ID NOS: 1, 3, 5, 7, and 9). In one embodiment the gene may be mutated, such as a deletion mutation. Alternatively the mutation can be a missense, or frame shift mutation. For example, if the mutation to be detected is a deletion mutation, the presence or absence of three nucleotides in this region.

The invention also relates to methods of detecting the presence or absence of dystonia disorder in a human wherein the dystonia disorder is characterized by one or more mutations in a dystonia gene.

Another aspect of the invention relates to methods of detecting the presence or absence of a dystonia disorder, wherein the test sample from the human is evaluated by performing a polymerase chain reaction, hereinafter “PCR,” with oligonucleotide primers capable of amplifying a dystonia gene. Following PCR amplification of a nucleic acid sample, the amplified nucleic acid fragments are separated and mutations in the tor-2 gene and alleles of the dystonia gene detected. For example, a mutation in the tor-2 gene is indicative of the presence of the torsion dystonia, whereas the lack of a mutation is indicative of a negative diagnosis.

An additional aspect of the invention is a method of determining the presence or absence of a dystonia disorder in a human including the steps of contacting a biological sample obtained from the human with a nucleic acid probe to a dystonia gene; maintaining the biological sample and the nucleic acid probe under conditions suitable for hybridization; detecting hybridization between the biological sample and the nucleic acid probe; and comparing the hybridization signal obtained from the human to a control sample which does or does not contain a dystonia disorder. The hybridization is performed with a nucleic acid fragment of a dystonia gene such as SEQ ID NOS: 1, 3, 5, 7, and 9. The nucleic acid probe can be labeled (e.g., fluorescent, radioactive, enzymatic, biotin label).

The invention also encompasses methods for predicting whether a human is likely to be affected with a dystonia disorder, comprising obtaining a biological sample from the human; contacting the biological sample with a nucleic acid probe; maintaining the biological sample and the nucleic acid probe under conditions suitable for hybridization; and detecting hybridization between the biological sample and the nucleic acid probe. In another embodiment the method further comprises performing PCR with oligonucleotide primers capable of amplifying a dystonia gene (e.g., SEQ ID NOS: 1, 3, 5, 7, and 9); and detecting a mutation in amplified DNA fragments of the dystonia gene, wherein the mutation in the dystonia gene is indicative of the presence or absence of the torsion dystonia. The hybridization can detect, for example, a deletion in nucleotides indicative of a positive diagnosis; or the presence of nucleotides indicative of a negative diagnosis.

The invention further provides for methods of determining the presence or absence of a dystonia disorder in a human comprising obtaining a biological sample from the human; and assessing the level of a dystonia protein in the biological sample comprising bodily fluids, tissues or both from the human. The levels or concentrations of the dystonia protein are determined by contacting the sample with at least one antibody specific to a dystonia protein, and detecting the levels of the dystonia protein. An alteration in the dystonia protein levels is indicative of a diagnosis. The antibody used in the method can be a polyclonal antibody or a monoclonal antibody and can be detectably labeled (e.g., fluorescence, biotin, colloidal gold, enzymatic). In another embodiment the method of assessing the level or concentration of the dystonia protein further comprises contacting the sample with a second antibody specific to the dystonia protein or a complex between an antibody and the dystonia protein.

The present invention also provides for a kit for diagnosing the presence or absence of a dystonia disorder in a human comprising one or more reagents for detecting a mutation in a dystonia gene, such as DYT1, or a dystonia protein, such as TOR-A, in a sample obtained from the human. The one or more reagents for detecting the torsion dystonia are used for carrying out an enzyme-linked immunosorbent assay or a radioimmunoassay to detect the presence of absence of dystonia protein. In another embodiment the kit comprises one or more reagents for detecting the torsion dystonia by carrying out a PCR, hybridization or sequence-based assay or any combination thereof.

It is also envisioned that the methods of the present invention can diagnosis a mutation in a dystonia gene, such as DYT1, which encodes a dystonia protein, such as TOR-A, wherein a mutation in the dystonia gene for the human is compared to a mutation in a dystonia gene for a parent of the human who is unaffected by a torsion dystonia, a parent of the human who is affected by the torsion dystonia and a sibling of the human who is affected by the torsin dystonia.

The invention also provides methods for therapeutic uses involving all or part of the nucleic acid sequence encoding torsin protein or torsin protein.

The invention further provides nucleic acid sequences useful as probes and primers for the identification of mutations or polymorphisms which mediate clinical neuronal diseases, or which confer increased vulnerability (e.g., genetic predisposition) respectively, to other neuronal diseases.

Another embodiment of the invention provides methods utilizing the disclosed probes and primers to detect mutations or polymorphisms in other neuronal genes implicated in conferring a particular phenotype which gives rise to overt clinical symptoms in a mammal that are consistent with (e.g., correlate with) the neuroanatomical expression of the gene. For example, the methods described herein can be used to confirm the role of TOR-1, TOR-2, ooc-5, TOR-A or TOR-B in neuronal diseases, including but not limited to dopamine-mediated diseases, movement disorders, neurodegenerative diseases, neurodevelopmental diseases and neuropsychiatric disorders.

An particular embodiment provides a method of identifying a gene comprising a mutation or a polymorphism resulting in a dopamine-mediated disease, or a neuronal disease. Examples of such diseases are represented in Table 1.

Another embodiment of the invention provides a method of identifying a mutation or polymorphism in a neuronal gene which confers increased susceptibility to a neuronal disease.

Another object of the present invention is a method of reducing, arresting, alleviating, ameliorating, or preventing protein aggregation in the presence of a torsin protein relative to a level of protein aggregation in the absence of the torsin protein. The torsin protein may be mutated. This method may be conducted in the presence of further compounds that of reducing, arresting, alleviating, ameliorating, or preventing protein aggregation

Another object of the present invention is a method of reducing, arresting, alleviating, ameliorating, or preventing cellular dysfunction as a result of protein aggregation. This method may be conducted in the presence of further compounds that of reducing, arresting, alleviating, ameliorating, or preventing cellular dysfunction as a result of protein aggregation.

Another object of the present invention is a method of treating, reducing, arresting, alleviating, ameliorating, or preventing protein-aggregation-associated diseases. Examples of protein-aggregation-associated diseases are those represented in Table 1. This method may be conducted in the presence of further compounds that of reducing, arresting, alleviating, ameliorating, or preventing protein-aggregation-associated diseases.

Another object of the present invention is a method of treating, reducing, arresting, alleviating, ameliorating, or preventing symptoms of protein-aggregation-associated diseases. Examples of protein-aggregation-associated diseases are those represented in Table 1. This method may be conducted in the presence of further compounds that of reducing, arresting, alleviating, ameliorating, or preventing symptoms of protein-aggregation-associated diseases.

### BRIEF DESCRIPTION OF THE FIGURES

FIG. 1: A polynucleotide sequence alignment of tor-2 vs. DYT1.

FIG. 2: A polynucleotide sequence alignment of tor-2 vs. DYT2.

FIG. 3: A polypeptide sequence alignment of TOR-1, TOR-2, OOC-5, TOR-A, and TOR-B.

FIG. 4a: Expression of 19 polyglutamine repeats (Q19).

FIG. 4b: Expression of 82 polyglutamine repeats (Q82).

FIG. 4c: Co-expression of Q82 and tor-2.

FIG. 4d: Co-expression of Q82 and tor-2/Δ368.

FIG. 5: Size of Q82 aggregates.

FIG. 6a: Tail pictures of Q82, Q82+tor-2, and Q82+tor-2/Δ368.

FIG. 6b: Close-up pictures of Q82, Q82+tor-2, and Q82+tor-2/Δ368.

FIG. 7: Graph of Q19 aggregate accumulation vs. time.

FIG. 8: Immunolocalization by whole worm antibody staining with tor-2-specific antibody.

FIG. 9. Western blot of whole protein extracts from C. elegans with actin control and tor-2 antibody.

FIG. 10a: Expression of 82 polyglutamine repeats (Q82).

FIG. 10b: Co-expression of Q82 and TOR-2.

FIG. 10c: Co-expression of Q82 and OOC-5.

FIG. 10d: Co-expression of Q82 and TOR-A.

FIG. 10e: Co-expression of Q82 and OOC-5 and TOR-2.

### DETAILED DESCRIPTION OF THE INVENTION

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausebel et al (eds.), John Wiley & Sons, New York (2001) and the various references cited therein.

Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting. The present invention provide torsin proteins and polynucleotides that encode the proteins. Torsin proteins are known to occur in humans and thought to occur C. elegans. Until now, the function of torsin proteins was completely unknown. However, the present invention establishes that at least one function of torsin proteins is the prevention of protein aggregation. There are two human torsin proteins, TOR1A and TOR1B, and there are three torsin proteins from C. elegans, TOR-1, TOR-2, and OOC-5.

Within the context of the present invention “isolated” or “purified” means separated out of its natural environment, which is also substantially free of other contaminating proteins, polynucleotides, and/or other biological materials often found in cell extracts.

Within the context of the present invention “Polynucleotide” in general relates to polyribonucleotides and polydeoxyribonucleotides, it being possible for these to be non-modified RNA or DNA or modified RNA or DNA.

“Consisting essentially of”, in relation to a nucleic acid sequence, is a term used hereinafter for the purposes of the specification and claims to refer to substitution of nucleotides as related to third base degeneracy. As appreciated by those skilled in the art, because of third base degeneracy, almost every amino acid can be represented by more than one triplet codon in a coding nucleotide sequence. Further, minor base pair changes may result in variation (conservative substitution) in the amino acid sequence encoded, are not expected to substantially alter the biological activity of the gene product. Thus, a nucleic acid sequencing encoding a protein or peptide as disclosed herein, may be modified slightly in sequence (e.g., substitution of a nucleotide in a triplet codon), and yet still encode its respective gene product of the same amino acid sequence. The amino acid sequence of TOR-2 is shown as SEQ ID NO:2 and the genomic sequence encoding the TOR-2 protein is shown as SEQ ID NO:1. The amino acid sequence of TOR-1 is shown as SEQ ID NO:4 and the genomic sequence encoding the TOR-1 protein is shown as SEQ ID NO:3. The amino acid sequence of OOC-5 is shown as SEQ ID NO:6 and the genomic sequence encoding the OOC-5 protein is shown as SEQ ID NO:5. The amino acid sequence of TOR-A is shown as SEQ ID NO:8 and the genomic sequence encoding the TOR-A protein is shown as SEQ ID NO:7. The amino acid sequence of TOR-B is shown as SEQ ID NO:10 and the genomic sequence encoding the TOR-B protein is shown as SEQ ID NO:9.

One skilled in the art will realize that organisms other than humans will also contain torsin genes (for example, eukaryotes; more specifically, mammals (preferably, gorillas, rhesus monkeys, and chimpanzees), rodents, worms (preferably, C. elegans), insects (preferably, D. melanogaster) birds, fish, yeast, and plants). The invention is intended to include, but is not limited to, torsin nucleic acid molecules isolated from the abovedescribed organisms.

Isolated nucleic acid molecules of the present invention are also meant to include those chemically synthesized. For example, a nucleic acid molecule with the nucleotide sequence which codes for the expression product of a torsin gene can be designed and, if necessary, divided into appropriate smaller fragments. Then an oligomer which corresponds to the nucleic acid molecule, or to each of the divided fragments, can be synthesized. Such synthetic oligonucleotides can be prepared synthetically (Matteucci et al., 1981, J. Am. Chem. Soc. 103:3185-3191) or by using an automated DNA synthesizer. An oligonucleotide can be derived synthetically or by cloning. If necessary, the 5′ ends of the oligonucleotides can be phosphorylated using T4 polynucleotide kinase. Kinasing the 5′ end of an oligonucleotide provides a way to label a particular oligonucleotide by, for example, attaching a radioisotope (usually .sup.32p) to the 5′ end. Subsequently, the oligonucleotide can be subjected to annealing and ligation with T4 ligase or the like.

To isolate the torsin genes or also other genes, a gene library is first set up. The setting up of gene libraries is described in generally known textbooks and handbooks. The textbook by Winnacker: Gene und Klone, Eine Einführung in die Gentechnologie [Genes and Clones, An Introduction to Genetic Engineering] (Verlag Chemie, Weinheim, Germany, 1990), or the handbook by Sambrook et al.: Molecular Cloning, A Laboratory Manual (Cold Spring Harbor Laboratory Press, 1989) may be mentioned as an example. A well-known gene library is that of the E. coli K-12 strain W3110 set up in λ vectors by Kobara et al. (Cell 50, 495-508 (1987)).

To prepare a gene library in E. coli, it is also possible to use plasmids such as pBR322 (Bolivar, 1979, Life Sciences, 25, 807-818) or pUC9 (Vieira et al., 1982, Gene, 19:259-268). Suitable hosts are, in particular, those E. coli strains which are restriction- and recombination-defective, such as the strain DH5αmcr, which has been described by Grant et al. (Proceedings of the National Academy of Sciences USA, 87 (1990) 4645-4649).

The long DNA fragments cloned with the aid of cosmids or other λ vectors can then in turn be subcloned and subsequently sequenced in the usual vectors which are suitable for DNA sequencing, such as is described e.g. by Sanger et al. (Proceedings of the National Academy of Sciences of the United States of America, 74:5463-5467, 1977).

The resulting DNA sequences can then be investigated with known algorithms or sequence analysis programs, such as e.g. that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new torsin sequences for the torsin genes which are related to SEQ ID NOS. 2, 4, 6, 8, and 10, is a constituent of the present invention has been found in this manner. The amino acid sequence of the corresponding protein has furthermore been derived from the present DNA sequence by the methods described above. The resulting amino acid sequence of the torsin gene products is shown in SEQ ID NOS. 2, 4, 6, 8, and 10.

Coding DNA sequences, which result from SEQ ID NOS. 1, 3, 5, 7, and 9 by the degeneracy of the genetic code, are also a constituent of the invention. In the same way, DNA sequences, which hybridize with SEQ ID NOS. 1, 3, 5, 7, and 9 or parts of SEQ ID NOS. 1, 3, 5, 7, and 9, are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. are of neutral function. It is furthermore known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences, which result in a corresponding manner from SEQ ID NOS. 2, 4, 6, 8, and 10, are also a constituent of the invention.

In the same way, DNA sequences, which hybridize with SEQ ID NOS. 1, 3, 5, 7, and 9 or parts of SEQ ID NOS. 1, 3, 5, 7, and 9, are a constituent of the invention. Finally, DNA sequences, which are prepared by the polymerase chain reaction (PCR) using primers, which result from SEQ ID NOS. 1, 3, 5, 7, and 9, are a constituent of the invention. Such oligonucleotides typically have a length of at least 15 nucleotides.

The skilled artisan will find instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (international Journal of Systematic Bacteriology 41: 255-260 (1991)). The hybridization takes place under stringent conditions, that is to say only hybrids in which the probe and target sequence, i.e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5×SSC buffer at a temperature of approx. 50° C.-68° C., for example, can be employed for the hybridization reaction. Probes can also hybridize here with polynucleotides, which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) a temperature of approx. 50° C.-68° C. being established. It is optionally possible to lower the salt concentration to 0.1×SSC. Polynucleotide fragments which are, for example, at least 70% or at least 80% or at least 90% to 95% identical to the sequence of the probe employed can be isolated by increasing the hybridization temperature stepwise from 50° C. to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

A skilled artisan will find instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

A “mutation” is any detectable change in the genetic material which can be transmitted to daughter cells and possibly even to succeeding generations giving rise to mutant cells or mutant individuals. If the descendants of a mutant cell give rise only to somatic cells in multicellular organisms, a mutant spot or area of cells arises. Mutations in the germ line of sexually reproducing organisms can be transmitted by the gametes to the next generation resulting in an individual with the new mutant condition in both its somatic and germ cells. A mutation can be any (or a combination of) detectable, unnatural change affecting the chemical or physical constitution, mutability, replication, phenotypic function, or recombination of one or more deoxyribonucleotides; nucleotides can be added, deleted, substituted for, inverted, or transposed to new positions with and without inversion. Mutations can occur spontaneously and can be induced experimentally by application of mutagens. A mutant variation of a nucleic acid molecule results from a mutation. A mutant polypeptide can result form a mutant nucleic acid molecule and also refers to a polypeptide which is modified at one, or more, amino acid residues from the wildtype (naturally occurring) polypeptide. The term “mutation”, as used herein, can also refer to any modification in a nucleic acid sequence encoding a dystonia protein. For example, the mutation can be a point mutation or the addition, deletion, insertion and/or substitution of one or more nucleotides or any combination thereof. The mutation can be a missense or frameshift mutation. Modifications can be, for example, conserved or non-conserved, natural or unnatural.

“Consisting essentially of”, in relation to amino acid sequence of a protein or peptide, is a term used hereinafter for the purposes of the specification and claims to refer to a conservative substitution or modification of one or more amino acids in that sequence such that the tertiary configuration of the protein or peptide is substantially unchanged.

“Conservative substitutions” is defined by aforementioned function, and includes substitutions of amino acids having substantially the same charge, size, hydrophilicity, and/or aromaticity as the amino acid replaced. Such substitutions, known to those of ordinary skill in the art, include glycine-alanine-valine; isoleucine-leucine; tryptophan-tyrosine; aspartic acid-glutamic acid; arginine-lysine; asparagine-glutamine; and serine-threonine. “modification”, in relation to amino acid sequence of a protein or peptide, is defined functionally as a deletion of one or more amino acids which does not impart a change in the conformation, and hence the biological activity, of the protein or peptide sequence.

The term “expression vector” refers to an polynucleotide that encodes the torsin proteins or fragments thereof of the invention and provides the sequences necessary for its expression in the selected host cell. The recombinant host cells of the present invention may be maintained in vitro, e.g., for recombinant protein, polypeptide or peptide production Equally, the recombinant host cells could be host cells in vivo, such as results from immunization of an animal or human with a nucleic acid segment of the invention. Accordingly, the recombinant host cells may be prokaryotic or eukaryotic host cells, such as E. coli, Saccharomyces cerevisiae or other yeast, mammalian or human host cells. Expression vectors will generally include a transcriptional promoter and terminator, or will provide for incorporation adjacent to an endogenous promoter. Expression vectors will usually be plasmids, further comprising an origin of replication and one or more selectable markers. However, expression vectors may alternatively be viral recombinants designed to infect the host, or integrating vectors designed to integrate at a preferred site within the host's genome. Examples of other expression vectors are disclosed in Molecular Cloning: A Laboratory Manual, Third Edition, Sambrook, Fritsch, and Maniatis, Cold Spring Harbor Laboratory Press, 2001. In a preferred embodiment these polynucleotides that hybridize under stringent conditions also encode a protein or peptide which has torsin activity.

“Torsin activity” within the context of the present invention includes reducing, alleviating, arresting, ameliorating, and inhibiting protein aggregation.

“Torsin gene” within the context of the present invention includes any polynucleotide encoding a polypeptide having torsin activity.

“Torsin protein” within the context of the present invention includes any polypeptide having torsin activity.

The common amino acids are generally known in the art. Additional amino acids that may be included in the peptide of the present invention include: L-norleucine; aminobutyric acid; L-homophenylalanine; L-norvaline; D-alanine; D-cysteine; D-aspartic acid; D-glutamic acid; D-phenylalanine; D-histidine; D-isoleucine; D-lysine; D-leucine; D-methionine; D-asparagine; D-proline; D-glutamine; D-arginine; D-serine; D-threonine; D-valine; D-tryptophan; D-tyrosine; D-ornithine; aminoisobutyric acid; L-ethylglycine; L-t-butylglycine; penicillamine; I-naphthylalanine; cyclohexylalanine; cyclopentylalanine; aminocyclopropane carboxylate; aminonorbornylcarboxylate; L-α-methylalanine; L-α-methylcysteine; L-α-methylaspartic acid; L-α-methylglutamic acid; L-α-methylphenylalanine; L-α-methylhistidine; L-α-methylisoleucine; L-α-methyllysine; L-α-methylleucine; L-α-methylmethionine; L-α-methylasparagine; L-α-methylproline; La-methylglutamine; L-α-methylarginine; L-α-methylserine; L-α-methylthreonine; L-α-methylvaline; L-α-methyltryptophan; L-α-methyltyrosine; L-α-methylomithine; L-α-methylnorleucine; amino-α-methylbutyric acid; L-α-methylnorvaiine; L-α-methylhomophenylalanie; L-α-methylethylglycine; methyl-α-aminobutyric acid; methylaminoisobutyric acid; L-α-methyl-t-butylglycine; methylpenicillamine; methyl-α-naphthylalanine; methylcyclohexylalanine; methylcyclopentylalanine; D-α-methylalanine; D-α-methylornithine; D-α-methylcysteine; D-α-methylaspartic acid; D-α-methylglutamic acid; D-α-methylphenylalanine; D-α-methylhistidine; D-α-methylisoleucine; D-α-methyllysine; D-α-methylleucine; D-α-methylmethionine; D-α-methylasparagine; D-α-methylproline; D-α-methylglutamine; D-α-methylarginine; D-α-methylserine; D-α-methylthreonine; D-α-methylvaline; D-α-methyltryptophan; D-α-methyltyrosine; L-N-methylalanine; L-N-methylcysteine; L-N-methylaspartic acid; L-N-methylglutamic acid; L-N-methylphenylalanine; L-N-methylhistidine; L-N-methylisoleucine; L-N-methyllysine; L,N-methylleucine; L-N-methylmethionine; L-N-methylasparagine; N-methylcyclohexylalanine; L-N-methylglutamine; L-N-methylarginine; L-N-methylserine; L-N-methylthreonine; L-N-methylvaline; L-N-methyltryptophan; L-N-methyltyrosine; L-N-methylomithine; L-N-methylnorleucine; N-amino-α-methylbutyric acid; L-N-methylnorvaline; L-N-methylhomophenylalanine; L-N-methylethylglycine; N-methyl-γaminobutyric acid; N-methylcyclopentylalanine; L-N-methyl-t-butylglycine; N-methylpenicillamine; N-methyl-α-naphthylalanine; N-methylaminoisobutyric acid; N-(2-aminoethyl)glycine; D-N-methylalanine; D-N-methylomithine; D-N-methylcysteine; D-N-methylaspartic acid; D-N-methylglutamic acid; D-N-methylphenylalanine; D-N-methylhistidine; D-N-methylisoleucine; D-N-methyllysine; D-N-methylleucine; D-N-methylmethionine; D-N-methylasparagine; D-N-methylproline; D-N-methylglutamine; D-N-methylarginine; D-N-methylserine; D-N-methylthreonine; D-N-methylvaline; D-N-methyltryptophan; D-N-methyltyrosine; N-methylglycine; N-(carboxymethyl)glycine; N-(2-carboxyethyl)glycine; N-benzylglycine; N-(imidazolylethyl)glycine; N-(1-methylpropyl)glycine; N-(4-aminobutyl)glycine; N-(2-methylpropyl)glycine; N-(2-methylthioethyl)glycine; N-(hydroxyethyl)glycine; N-(carbamylmethyl)glycine; N-(2-carbamylethyl)glycine; N-(1-methylethyl)glycine; N-(3-guanidinopropyl)glycine; N-(3-indolylethyl)glycine; N-(p-hydroxyphenethyl)glycine; N-(1-hydroxyethyl)glycine; N-(thiomethyl)glycine; N-(3-aminopropyl)glycine; N-cyclopropylglycine; N-cyclobutyglycine; N-cyclohexylglycine; N-cycloheptylglycine; N-cyclooctylglycine; N-cyclodecylglycine; N-cycloundecylglycine; N-cyclododecylglycine; N-(2,2-diphenylethyl)glycine; N-(3,3-diphenylpropyl)glycine; N-(N-(2,2-diphenylethyl)carbamylmethyl)glycine; N-(N-(3,3-diphenylpropyl)carbamylmethyl)glycine; and 1-carboxy-1-(2,2-diphenylethylamino)cyclopropane.

Because its amino acid sequence has been disclosed by the present invention, the TOR-1 and TOR-2 proteins or fragments thereof of the present invention can be produced by a known chemical synthesis method (for example, a liquid phase synthesis method, a solid phase synthesis method, and others; Izumiya. N., Kato. T., Aoyagi. H. Waki. M., “Basis and Experiments of Peptide Synthesis”, 1985, Maruzen Co., Ltd.) based on that sequence. Typically, peptide synthesis is carried out for shorter peptide fragments of about 100 amino acids or less.

The TOR-1 and TOR-2 proteins or fragments thereof of the present invention may contain one or more protected amino acid residues. The protected amino acid is an amino acid whose functional group or groups is/are protected with a protecting group or groups by a known method and various protected amino acids are commercially available.

The TOR-1 and TOR-2 proteins or fragments thereof of the present invention may be provided in a glycosylated as well as an unglycosylated form. Preparation of glycosylated TOR-1 and TOR-2 proteins or fragments thereof is known in the art and typically involves expression of the recombinant DNA encoding the peptide in a eukaryotic cell. Likewise, it is generally known in the art to express the recombinant DNA encoding the peptide in a prokaryotic (e.g., bacterial) cell to obtain a peptide, which is not glycosylated. These and other methods of altering carbohydrate moieties on glycoproteins is found, inter alia, in Essentials of Glycobiology (1999), Edited By Ajit Varki, Cold Spring Harbor Laboratory Press, Cold Spring Harbor, N.Y., the contents of which are incorporated herein by reference.

Alternatively, the TOR-1 and TOR-2 proteins or fragments thereof of the present invention can be produced by producing a polynucleotide (DNA or RNA) which corresponds to the amino acid sequence of the TOR-1 and TOR-2 proteins or fragments thereof of the present invention and producing the TOR-1 and TOR-2 proteins or fragments thereof by a genetic engineering technique using the polynucleotide. Polynucleotide coding sequences for amino acid residues are known in the art and are disclosed for example in Molecular Cloning: A Laboratory Manual, Third Edition, Sambrook, Fritsch, and Maniatis, Cold Spring Harbor Laboratory Press, 2001.

In another embodiment, the present invention relates to a purified polypeptide preferably, substantially pure) having an amino acid sequence corresponding to a torsin protein, or a functional derivative thereof. In a preferred embodiment, the polypeptide has the amino acid sequence set forth in SEQ ID NOS: 2, 4, 6, 8, and 10 or mutant or species variation thereof, or at least 70% identity, further at least 80% identity or and even further at least 90% identity thereof (preferably, at least 90%, 95%, 96%, 97%, 98%, or 99% identity or at least 95%, 96%, 97%, 98%, or 99% similarity thereof), or at least 6 contiguous amino acids thereof (preferably, at least 10, 15, 20, 25, or 50 contiguous amino acids thereof).

In a preferred embodiment, the invention relates to torsin epitopes. The epitope of these polypeptides is an immunogenic or antigenic epitope. An immunogenic epitope is that part of the protein which elicits an antibody response when the whole protein is the immunogen. An antigenic epitope is a fragment of the protein which can elicit an antibody response. Methods of selecting antigenic epitope fragments are well known in the art (Sutcliffe et al., 1983, Science. 219:660-666). Antigenic epitope-bearing peptides and polypeptides of the invention are useful to raise an immune response that specifically recognizes the polypeptides. Antigenic epitope-bearing peptides and polypeptides of the invention comprise at least 7 amino acids (preferably, 9, 10, 12, 15 or 20 amino acids) of the proteins of the Amino acid sequence variants of torsin can be prepared by mutations in the DNA. Such variants include, for example, deletions from, or insertions or substitutions of, residues within the amino acid sequence shown in SEQ ID NOS: 2, 4, 6, 8, and 10. Any combination of deletion, insertion, and substitution can also be made to arrive at the final construct, provided that the final construct possesses the desired activity.

While the site for introducing an amino acid sequence variation is predetermined, the mutation itself need not be predetermined. For example, to optimize the performance of a particular polypeptide with respect to a desired activity, random mutagenesis can be conducted at a target codon or region of the polypeptide, and the expressed variants can be screened for the optimal desired activity. Techniques for making substitution mutations at predetermined sites in DNA having a known sequence are well known, e.g., site-specific mutagenesis.

Preparation of a torsin variant in accordance herewith is preferably achieved by site-specific mutagenesis of DNA that encodes an earlier prepared variant or a non-variant version of the protein. Site-specific mutagenesis allows the production of torsin variants through the use of specific oligonucleotide sequences that encode the DNA sequence of the desired mutation. In general, the technique of site-specific mutagenesis is well known in the art (Adelman et al., 1983, DNA 2:183; Ausubel, et al., In: Current Protocols in Molecular Biology, John Wiley & Sons, (1998)).

Amino acid sequence deletions generally range from about 1 to 30 residues, more preferably 1 to 10 residues.

Amino acid sequence insertions include amino and/or carboxyl terminal fusions from one residue to polypeptides of essentially unrestricted length, as well as intrasequence insertions of single or multiple amino acid residues. Intrasequence insertions, (i.e., insertions within the complete torsin sequence) can range generally from about 1 to 10 residues, more preferably 1 to 5.

The third group of variants are those in which at least one amino acid residue in the torsin molecule, and preferably, only one, has been removed and a different residue inserted in its place.

Substantial changes in functional or immunological identity are made by selecting substitutions that are less conservative, i.e., selecting residues that differ more significantly in their effect on maintaining a) the structure of the polypeptide backbone in the area of the substitution, for example, as a sheet or helical conformation, b) the charge or hydrophobicity of the molecule at the target site, or c) the bulk of the side chain. The substitutions that in general are expected are those in which a) glycine and/or proline is substituted by another amino acid or is deleted or inserted; b) a hydrophilic residue, e.g., seryl or threonyl, is substituted for a hydrophobic residue, e.g., leucyl, isoleucyl, phenylalanyl, valyl, or alanyl; c) a cysteine residue is substituted for any other residue; d) a residue having an electropositive side chain, e.g., lysyl, arginyl, or histidyl, is substituted for a residue having an electronegative charge, e.g., glutamyl or aspartyl; or e) a residue having a bulky side chain, e.g., phenylalanine, is substituted for one not having such a side chain, e.g., glycine.

Some deletions, insertions and substitutions are not expected to produce radical changes in the characteristics of torsin. However, while it is difficult to predict the exact effect of the deletion, insertion or substitution in advance, one skilled in the art will appreciate that the effect can be evaluated by biochemical and in vivo screening assays. For example, a variant typically is made by site-specific mutagenesis of the native torsin-encoding nucleic acid, expression of the variant nucleic acid in cell culture, and, optionally, purification from the cell culture, for example, by immunoaffinity adsorption on a column (to absorb the variant by binding it to at least one immune epitope). The activity of the cell culture lysate or purified torsin variant is then screened by a suitable screening assay for the desired characteristic. For example, a change in the immunological character of the torsin molecule, such as affinity for a given antibody, can be measured by a competitive type immunoassay. Changes in immunomodulation activity can be measured by the appropriate assay. Modifications of such protein properties as redox or thermal stability, enzymatic activity, hydrophobicity, susceptibility to proteolytic degradation or the tendency to aggregate with carriers or into multimers are assayed by methods well known to those of ordinary skill in the art.

A variety of methodologies known in the art can be utilized to obtain the polypeptide of the present invention. In one embodiment, the polypeptide is purified from tissues or cells which naturally produce the peptide. Alternatively, the above described isolated nucleic acid fragments can be used to express the torsin protein in any organism. The samples of the present invention include cells, protein extracts or membrane extracts of cells, or biological fluids. The sample will vary based on the assay format, the detection method and the nature of the tissues, cells or extracts used as the sample

Any organism can be used as a source for the polypeptide of the invention, as long as the source organism naturally contains such a peptide. As used herein, “source organism” refers to the original organism from which the amino acid sequence of the polypeptide is derived, regardless of the organism the polypeptide is expressed in and ultimately isolated from.

One skilled in the art can readily follow known methods for isolating proteins in order to obtain the polypeptide free of natural contaminants. These include, but are not limited to: immunochromotography, size-exclusion chromatography, ion-exchange chromatography, hydrophobic interaction chromatography, and non-chromatographic separation methods.

In a preferred embodiment, the purification procedures comprise ion-exchange chromatography and size exclusion chromatography. Any of a large number of ion-exchange resins known in the art can be employed, including, for example, monoQ, Sepharose-Q, macro-prepQ, AG1-X2, or HQ. Examples of suitable size exclusion resins include, but are not limited to, Superdex 200, Superose 12, and Sephycryl 200. Elution can be achieved with aqueous solutions of potassium chloride or sodium chloride at concentrations ranging from 0.01 M to 2. OM over a wide range of pH.

In another embodiment, the present invention relates to a nucleic acid probe for the specific detection of the presence of torsin nucleic acid in a sample comprising the above-described nucleic acid molecules or at least a fragment thereof which hybridizes under stringent hybridization and wash conditions to torsin nucleic acid.

In one preferred embodiment, the present invention relates to an isolated nucleic acid probe consisting of 10 to 1000 nucleotides (preferably, 10 to 500, 10 to 100, 10 to 50, 10 to 35, 20 to 1000, 20 to 500, 20 to 100, 20 to 50, or 20 to 35) which hybridizes preferentially to torsin RNA or DNA, wherein said nucleic acid probe is or is complementary to a nucleotide sequence consisting of at least 10 consecutive nucleotides (preferably, 15, 18, 20, 25, or 30) from the nucleic acid molecule comprising a polynucleotide sequence at least 90% identical to one or more of the following: a nucleotide sequence encoding a torsin polypeptide (for example, those described by SEQ ID NOS: 2, 4, 6, 8, and 10); a nucleotide sequence complementary to any of the above nucleotide sequences; and any nucleotide sequence as previously described above.

The nucleic acid probe can be used to probe an appropriate chromosomal or cDNA library by usual hybridization methods to obtain another nucleic acid molecule of the present invention. A chromosomal DNA or cDNA library can be prepared from appropriate cells according to recognized methods in the art (Sambrook, J., Fritsch, E. F., and Maniatis, T., 1989, In: Molecular Cloning. A Laboratory Manual., Cold Spring Harbor Laboratory Press, Cold Spring Harbor).

In the alternative, chemical synthesis is carried out in order to obtain nucleic acid probes having nucleotide sequences which correspond to N-terminal and C-terminal portions of the torsin amino acid sequence. Thus, the synthesized nucleic acid probes can be used as primers in a polymerase chain reaction (PCR) carried out in accordance with recognized PCR techniques (PCR Protocols, A Guide to Methods and Applications, edited by Michael et al., Academic Press, 1990), utilizing the appropriate chromosomal, cDNA or cell line library to obtain the fragment of the present invention.

The hybridization probes of the present invention can be labeled for detection by standard labeling techniques such as with a radiolabeling, fluorescent labeling, biotin-avidin labeling, chemiluminescence, and the like. After hybridization, the probes can be visualized using known methods.

The nucleic acid probes of the present invention include RNA, as well as DNA probes, such probes being generated using techniques known in the art.

In one embodiment of the above described method, a nucleic acid probe is immobilized on a solid support. Examples of such solid supports include, but are not limited to, plastics such as polycarbonate, complex carbohydrates such as agarose and sepharose, and acrylic resins such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The sample used in the described methods will vary based on the assay format, the detection method and the nature of the tissues, cells or extracts used in the assay. Methods for preparing nucleic acid extracts of cells are well known in the art and can be readily adapted in order to obtain a sample which is compatible with the method utilized.

In another embodiment, the present invention relates to a method of detecting the presence of torsin nucleic acid in a sample by contacting the sample with the above-described nucleic acid probe, under specific hybridization conditions such that hybridization occurs, and detecting the presence of the probe bound to the nucleic acid molecule. One skilled in the art would select the nucleic acid probe according to techniques known in the art as described above. Samples to be tested include but should not be limited to RNA or DNA samples from human tissue.

In another embodiment, the present invention relates to a kit for detecting, in a sample, the presence of a torsin nucleic acid. The kit comprises at least one container having disposed therein the above-described nucleic acid probe. In a preferred embodiment, the kit further comprises other containers comprising wash reagents and/or reagents capable of detecting the presence of the hybridized nucleic acid probe. Examples of detection reagents include, but are not limited to radiolabeled probes, enzymatic probes (horseradish peroxidase, alkaline phosphatase), and affinity labeled probes (biotin, avidin, or streptavidin).

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris buffers, and the like), and containers which contain the reagents used to detect the hybridized probe, bound antibody, amplified product, or the like.

One skilled in the art will readily recognize that the nucleic acid probes described in the present invention can readily be incorporated into one of the established kit formats which are well known in the art.

In another embodiment, the present invention relates to a recombinant DNA molecule comprising, 5′ to 3′, a promoter effective to initiate transcription in a host cell and the above-described nucleic acid molecules. In another embodiment, the present invention relates to a recombinant DNA molecule comprising a vector and an above-described nucleic acid molecule.

In another embodiment, the present invention relates to a nucleic acid molecule comprising a transcriptional control region functional in a cell, a sequence complementary to an RNA sequence encoding an amino acid sequence corresponding to the above-described polypeptide, and a transcriptional termination region functional in the cell.

Preferably, the above-described molecules are isolated and/or purified DNA molecules.

In another embodiment, the present invention relates to a cell or non-human organism that contains an above-described nucleic acid molecule.

In another embodiment, the peptide is purified from cells which have been altered to express the peptide.

As used herein, a cell is said to be “altered to express a desired peptide” when the cell, through genetic manipulation, is made to produce a protein which it normally does not produce or which the cell normally produces at low levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression. The precise nature of the regulatory regions needed for gene expression can vary from organism to organism, but shall in general include a promoter region which, in prokaryotes for example, contains both the promoter, which directs the initiation of RNA transcription, as well as the DNA sequences that, when transcribed into RNA, will signal translational initiation. Such regions will normally include those 5′ non-coding sequences involved with initiation of transcription and translation, such as the TATA box, capping sequence, CAAT sequence, and the like.

If desired, the non-coding region 3′ to the torsin coding sequence can be obtained by the above-described methods. This region can be retained for its transcriptional termination regulatory sequences, such as termination and polyadenylation signals. Thus, by retaining the 3′ region naturally contiguous to the DNA sequence encoding a torsin gene, the transcriptional termination signals are provided. Where the transcriptional termination signals are not functional in the expression host cell, then a functional 3′ region derived from host sequences can be substituted.

Two DNA sequences (such as a promoter region sequence and an torsin coding sequence) are said to be operably linked if the nature of the linkage between the two DNA sequences does not (1) result in the introduction of a frameshift mutation, (2) interfere with the ability of the promoter region to direct the transcription of a torsin coding sequence, or (3) interfere with the ability of the torsin coding sequence to be transcribed by the promoter. Thus, a promoter region would be operably linked to a DNA sequence if the promoter were capable of effecting transcription of that DNA sequence.

The present invention encompasses the expression of the torsin coding sequence (or a functional derivative thereof) in either prokaryotic or eukaryotic cells. Prokaryotic hosts are, generally, the most efficient and convenient for the production of recombinant proteins. Prokaryotes most frequently are represented by various strains of E. coli, however other microbial strains can also be used, including other bacterial strains such as those belonging to bacterial families such as Bacillus, Streptomyces, Pseudomonas, Salmonella, Serratia, and the like. In prokaryotic systems, plasmid vectors that contain replication sites and control sequences derived from a species compatible with the host can be used. Examples of suitable plasmid vectors include pBR322, pUC18, pUC19, pUC118, pUC119 and the like; suitable phage or bacteriophage vectors include .lambdagt10, .lambda.gt11 and the like. For eukaryotic expression systems, suitable viral vectors include pMAM-neo, pKRC and the like. Preferably, the selected vector of the present invention has the capacity to replicate in the selected host cell.

To express torsin in a prokaryotic cell, it is necessary to operably link the torsin coding sequence to a functional prokaryotic promoter. Such promoters can be either constitutive or, more preferably, regulatable (i.e., inducible or derepressible). Examples of constitutive promoters include the in promoter of bacteriophage .lambda, the bla promoter of the .beta.-lactamase gene, and the CAT promoter of the chloramphenicol acetyl transferase gene, and the like. Examples of inducible prokaryotic promoters include the major right and left promoters of bacteriophage lambda. (P.sub.L and P.sub.R), the trp, recA, lacZ lacI, and gal promoters of E. coli, the .alpha-amylase (Ulmanen et al., 1985, J. Bacteriol. 162:176-182) and the .zeta.-28-specific promoters of B. subtilis (Gilman et al., 1984, Gene sequence 32:11-20), the promoters of the bacteriophages of B. subtilis (Gryczan, In: The Molecular Biology of the Bacilli, Academic Press, Inc., N.Y. (1982)), and Streptomyces promoters (Ward, et al., 1986, Mol. Gen. Genet. 203:468-478).

Proper expression in a prokaryotic cell also requires the presence of a ribosome binding site upstream of the gene sequence-encoding sequence (Gold et al., 1981, Ann. Rev. Microbiol. 35:365-404).

The selection of control sequences, expression vectors, transformation methods, and the like, is dependent on the type of host cell used to express the gene. The terms “transformants” or “transformed cells” include the primary subject cell and cultures derived therefrom, without regard to the number of transfers. It is also understood that all progeny cannot be precisely identical in DNA content, due to deliberate or inadvertent mutations. However, as defined, mutant progeny have the same functionality as that of the originally transformed cell.

Host cells which can be used in the expression systems of the present invention are not strictly limited, provided that they are suitable for use in the expression of the torsin peptide of interest. Suitable hosts include eukaryotic cells. Preferred eukaryotic hosts include, for example, yeast, fungi, insect cells, mammalian cells either in vivo, or in tissue culture. Preferred mammalian cells include HeLa cells, cells of fibroblast origin such as VERO or CHO-K1, or cells of lymphoid origin and their derivatives.

In addition, plant cells are also available as hosts, and control sequences compatible with plant cells, such as the cauliflower mosaic virus 35S and 19S, nopaline synthase promoter and polyadenylation signal sequences are available.

Another preferred host is an insect cell, for example Drosophila melanogaster larvae. Using insect cells as hosts, the Drosophila alcohol dehydrogenase promoter can be used (Rubin, 1988, Science. 240:1453-1459). Alternatively, baculovirus vectors can be engineered to express large amounts of torsin protein in insect cells (Jasny, 1987, Science. 238:1653; Miller et al., In: Genetic Engineering (1986), Setlow, J. K., et al., Eds., Plenum, Vol. 8, pp. 277-297).

Another example of a host cell is that of within C. elegans. Examples of controlling expression within C. elegans include RNA interference (RNAi). Fire et al. have described that feeding C. elegans polynucleotides similar to that of the gene to be expressed can result in the attenuation of that gene's expression. The literature is full of references describing the many methods to control the expression of a gene through RNAi (See for example, U.S. Pat. Nos. 6,355,415, 6,326,193, 6,278,039, 6,274,630, 6,266,560, 6,255,071, 6,190,867, 6,025,192, 5,837,503, 5,726,299, 5,714,323, 5,693,781, 5,616,459, 5,565,333, 5,418,149, 5,198,346, 5,096,815, and 5,015,573).

Different host cells have characteristic and specific mechanisms for the translational and post-translational processing and modification (e.g., glycosylation and cleavage) of proteins. Appropriate cell lines or host systems can be chosen to ensure the desired modification of the foreign protein expressed.

Any of a series of yeast gene expression systems can be utilized which incorporate promoter and termination elements from the actively expressed gene sequences coding for glycolytic enzymes. These enzymes are produced in large quantities when yeast are grown in mediums rich in glucose. Known glycolytic gene sequences can also provide very efficient transcriptional control signals.

Yeast provides substantial advantages over prokaryotes in that it can perform post-translational peptide modifications. A number of recombinant DNA strategies exist which utilize strong promoter sequences and high copy number of plasmids which can be utilized for production of the desired proteins in yeast. Yeast recognizes leader sequences on cloned mammalian gene products and secretes peptides bearing leader sequences (i.e., pre-peptides).

For a mammalian host, several possible vector systems are available for the expression of torsin. A wide variety of transcriptional and translational regulatory sequences can be employed, depending upon the nature of the host. The transcriptional and translational regulatory signals can be derived from viral sources, such as adenovirus, bovine papilloma virus, simian virus, or the like, where the regulatory signals are associated with a particular gene which has a high level of expression. Alternatively, promoters from mammalian expression products, such as actin, collagen, myosin, and the like, can be employed. Transcriptional initiation regulatory signals can be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of torsin in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer, et al., 1982, J. Mol. Appl. Gen. 1:273-288); the TK promoter of herpes virus (McKnight, 1982, Cell. 31:355-365); the SV40 early promoter (Benoist, et al., 1981, Nature. 290:304-310); the yeast ga14 gene promoter (Johnston, et al., 1982, Proc. Nat. Acad. Sci. USA 79:6971-6975; Silver, et al., 1984, Proc. Natl. Acad. Sci. USA 81:595 1 5955) and the CMV immediate-early gene promoter (Thomsen, et al., 1984, Proc. Natl. Acad. Sci. USA 81:659-663).

As is widely known, translation of eukaryotic mRNA is initiated at a codon which encodes methionine. For this reason, it is preferable to ensure that the linkage between a eukaryotic promoter and a torsin coding sequence does not contain any intervening codons which are capable of encoding a methionine (i.e., AUG). The presence of such codons results either in a formation of a fusion protein (if the AUG codon is in the same reading frame as the torsin coding sequence) or a frame-shift mutation (if the AUG codon is not in the same reading frame as the torsin coding sequence).

A torsin nucleic acid molecule and an operably linked promoter can be introduced into a recipient prokaryotic or eukaryotic cell either as a non-replicating DNA (or RNA) molecule, which can either be a linear molecule or, more preferably, a closed covalent circular molecule. Since such molecules are incapable of autonomous replication, the expression of the gene can occur through the transient expression of the introduced sequence. Alternatively, permanent expression can occur through the integration of the introduced DNA sequence into the host chromosome

In one embodiment, a vector is employed which is capable of integrating the desired gene sequences into the host cell chromosome. Cells which have stably integrated the introduced DNA into their chromosomes can be selected on the basis of one or more markers which allow for selection of host cells which contain the expression vector. Such markers can provide, for example, for autotrophy to an auxotrophic host or for biocide resistance, e.g., to antibiotics or to heavy metal poisoning, such as by copper, or the like. The selectable marker gene sequence can either be contained on the vector of the DNA gene to be expressed, or introduced into the same cell by co-transfection. Additional elements might also be necessary for optimal synthesis of mRNA. These elements can include splice signals, as well as transcription promoters, enhancer signal sequences, and termination signals. cDNA expression vectors incorporating such elements have been described (Okayama, 1983, Molec. Cell Biol. 3:280).

In a preferred embodiment, the introduced nucleic acid molecule will be incorporated into a plasmid or viral vector capable of autonomous replication in the recipient host. Any of a wide variety of vectors can be employed for this purpose. Factors of importance in selecting a particular plasmid or viral vector include: the ease with which recipient cells that contain the vector can be recognized and selected from those recipient cells that do not contain the vector, the desired number of copies of the vector present in the host cell; and the ability to “shuttle” the vector between host cells of different species, i.e., between mammalian cells and bacteria. Preferred prokaryotic vectors include plasmids such as those capable of replication in E. coli (for example, pBR322, Co1E1, pSC101, pACYC 184, and .pi.VX). Such plasmids are commonly known to those of skill in the art (Sambrook, J., Fritsch, E. F., and Maniatis, T., 1989, In: Molecular Cloning. A Laboratory Manual., Cold Spring Harbor Laboratory Press, Cold Spring Harbor). B. subtilis derived plasmids include pC194, pC221, pT127, and the like (Gryczan, In: The Molecular Biology of the Bacilli, Academic Press, NY (1982), pp. 307-329). Suitable Streptomyces plasmids include pIJ101 (Kendall, et al., 1987, J. Bacteriol. 169:4177-4183), and streptomyces bacteriophages such as .phi.C31 (Chater, et al., In: Sixth International Symposium on Actinomycetales Biology, Akademiai Kaido, Budapest, Hungary (1986), pp. 45-54). Pseudomonas plasmids have also been described (John, et al., 1986, Rev. Infect. Dis. 8:693-704; Izaki, 1978, Jpn. J. Bacteriol. 33:729-742).

Preferred eukaryotic plasmids include, for example, BPV, vaccinia, SV40, 2 mu. circle, and the like, or their derivatives. Such plasmids are well known in the art (Botstein, et al., 1982, Miami Wntr. Symp. 19:265-274; Broach, hi: The Molecular Biology of the Yeast Saccharomyces: Life Cycle and Inheritance, Cold Spring Harbor Laboratory, Cold Spring Harbor, N.Y., p. 445-470 (1981); Broach, 1982, Cell. 28:203-204; Bollon, et al, 1980, J. Clin. Hematol. Oncol. 10:39-48; Maniatis, In: Cell Biology: A Comprehensive Treatise, Vol. 3, Gene Sequence Expression, Academic Press, NY, pp. 563-608 (1980)).

Once the vector or nucleic acid molecule containing the construct has been prepared for expression, the DNA construct can be introduced into an appropriate host cell by any of a variety of suitable means, i.e., transformation, transfection, lipofection, conjugation, protoplast fusion, electroporation, particle gun technology, calcium phosphate precipitation, direct microinjection, and the like. After the introduction of the vector, recipient cells are grown in a selective medium that allows for selection of vector containing cells. Expression of the cloned gene results in the production of torsin. This can take place in the transformed cells as such, or following the induction of these cells to differentiate (for example, by administration of bromodeoxyuracil to neuroblastoma cells or the like).

In another embodiment, the present invention relates to an antibody having binding affinity specifically to a torsin polypeptide as described above or specifically to a torsin polypeptide binding fragment thereof. An antibody binds specifically to a torsin polypeptide or binding fragment thereof if it does not bind to non-torsin polypeptides. Those which bind selectively to torsin would be chosen for use in methods which could include, but should not be limited to, the analysis of altered torsin expression in tissue containing torsin.

The torsin proteins of the present invention can be used in a variety of procedures and methods, such as for the generation of antibodies, for use in identifying pharmaceutical compositions, and for studying DNA/protein interaction.

The torsin peptide of the present invention can be used to produce antibodies or hybridomas. One skilled in the art will recognize that if an antibody is desired, such a peptide would be generated as described herein and used as an immunogen.

The antibodies of the present invention include monoclonal and polyclonal antibodies, as well as fragments of these antibodies. The invention further includes single chain antibodies. Antibody fragments which contain the idiotype of the molecule can be generated by known techniques. For example, such fragments include but are not limited to: the F(ab′).sub.2 fragment; the Fab′ fragments, Fab fragments, and Fv fragments.

Of special interest to the present invention are antibodies to torsin which are produced in humans, or are “humanized” (i.e., non-immunogenic in a human) by recombinant or other technology. Humanized antibodies can be produced, for example by replacing an immunogenic portion of an antibody with a corresponding, but non-immunogenic portion (i.e., chimeric antibodies (Robinson, R. R., et al., International Patent Publication PCT/US86/02269; Akira, K., et al., European Patent Application 184,187; Taniguchi, M., European Patent Application 171,496; Morrison, S. L., et al., European Patent Application 173,494; Neuberger, M. S., et al., PCT Application WO 86/01533; Cabilly, S., et al., European Patent Application 125,023; Better, M., et al, 1988, Science. 240:1041-1043; Liu, A. Y., et al., 1987, Proc. Natl. Acad. Sci. USA. 84:3439-3443; Liu, A. Y., et al., 1987, J. Immunol. 139:3521-3526; Sun, L. K., et al., 1987, Proc. Natl. Acad. Sci. USA 84:214-218; Nishimura, Y., et al., 1987, Canc. Res. 47:999-1005; Wood, C. R., et al., 1985, Nature. 314:446-449); Shaw, et al., 1988, J. Natl. Cancer Inst. 80:1553-1559) and “humanized” chimeric antibodies (Morrison, S. L., 1985, Science. 229:1202-1207; Oi, V. T., et al., 1986, BioTechniques 4:214)). Suitable “humanized” antibodies can be alternatively produced by CDR or CEA substitution (Jones, P. T., et al., 1986, Nature. 321:552-525; Verhoeyan, et al., 1988, Science. 239:1534; Beidler, C. B., et al., 1988, J. Immunol. 141:4053-4060).

In another embodiment, the present invention relates to a hybridoma which produces the above-described monoclonal antibody. A hybridoma is an immortalized cell line which is capable of secreting a specific monoclonal antibody.

In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands (1984); St. Groth, et al., 1980, J. Immunol. Methods. 35:1-21).

The inventive methods utilize antibodies reactive with torsin proteins or portions thereof. In a preferred embodiment, the antibodies specifically bind with torsin proteins or a portion or fragment thereof. The antibodies can be polyclonal or monoclonal, and the term antibody is intended to encompass polyclonal and monoclonal antibodies, and functional fragments thereof. The terms polyclonal and monoclonal refer to the degree of homogeneity of an antibody preparation, and are not intended to be limited to particular methods of production.

Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide can be modified or administered in an adjuvant in order to increase the peptide antigenicity. Methods of increasing the antigenicity of a polypeptide are well known in the art. Such procedures include coupling the antigen with a heterologous protein (such as globulin or .beta.-galactosidase) or through the inclusion of an adjuvant during immunization.

For monoclonal antibodies, spleen cells from the immunized animals are removed, fused with myeloma cells, and allowed to become monoclonal antibody producing hybridoma cells.

Any one of a number of methods well known in the art can be used to identify the hybridoma cell which produces an antibody with the desired characteristics. These include screening the hybridomas by an ELISA assay, Western blot analysis, or radioimmunoassay (Lutz, et al., 1988, Exp. Cell Res. 175:109-124).

Hybridomas secreting the desired antibodies are cloned and the class and subclass is determined using procedures known in the art (Campbell, In: Monoclonal Antibody Technology. Laboratory Techniques in Biochemistry and Molecular Biology, supra (1984)).

For polyclonal antibodies, antibody containing antisera is isolated from the immunized animal and is screened for the presence of antibodies with the desired specificity using one of the above-described procedures.

In another embodiment of the present invention, the above-described antibodies are detectably labeled. Antibodies can be detectably labeled through the use of radioisotopes, affinity labels (such as biotin, avidin, and the like), enzymatic labels (such as horse radish peroxidase, alkaline phosphatase, and the like) fluorescent labels (such as FITC or rhodamine, and the like), paramagnetic atoms, and the like. Procedures for accomplishing such labeling are well-known in the art (Stemberger, et al., 1970, J. Histochem. Cytochem. 18:315; Bayer, et al., 1979, Meth. Enzym. 62:308; Engval, et al., 1972, Immunol. 109:129; Goding, 1976, J. Immunol. Meth. 13:215). The labeled antibodies of the present invention can be used for in vitro, in vivo, and in situ assays to identify cells or tissues which express a specific peptide.

In another embodiment of the present invention the above-described antibodies are immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir, et al., In: “Handbook of Experimental Imunology,” 4th Ed., Blackwell Scientific Publications, Oxford, England, Chapter 10 (1986); Jacoby, et al., 1974, Meth. Enzym. Vol. 34. Academic Press, N.Y.). The immobilized antibodies of the present invention can be used for in vitro, in vivo, and in situ assays as well as in immunochromatography.

Furthermore, one skilled in the art can readily adapt currently available procedures, as well as the techniques, methods and kits disclosed above with regard to antibodies, to generate peptides capable of binding to a specific peptide sequence in order to generate rationally designed antipeptide peptides (Hurby, et al., In: “Application of Synthetic Peptides: Antisense Peptides,” In Synthetic Peptides, A User's Guide, W.H. Freeman, N.Y., pp. 289-307 (1992); Kaspczak, et al., 1989, Biochemistry 28:9230-9238).

Anti-peptide peptides can be generated in one of two fashions. First, the anti-peptide peptides can be generated by replacing the basic amino acid residues found in the torsin peptide sequence with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine

In another embodiment, the present invention relates to a method of detecting a torsin polypeptide in a sample, comprising: contacting the sample with an above-described antibody (or protein), under conditions such that immunocomplexes form, and detecting the presence of the antibody bound to the polypeptide. In detail, the methods comprise incubating a test sample with one or more of the antibodies of the present invention and assaying whether the antibody binds to the test sample. Altered levels of torsin in a sample as compared to normal levels can indicate a specific disease.

In a further embodiment, the present invention relates to a method of detecting a torsin antibody in a sample, comprising: contacting the sample with an above-described torsin protein, under conditions such that immunocomplexes form, and detecting the presence of the protein bound to the antibody or antibody bound to the protein. In detail, the methods comprise incubating a test sample with one or more of the proteins of the present invention and assaying whether the antibody binds to the test sample.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ the antibodies of the present invention (Chard, In: An Introduction to Radioimmunoassay and Related Techniques, Elsevier Science Publishers, Amsterdam, The Netherlands (1986); Bullock, et al., In: Techniques in Immunocytochemistry, Academic Press, Orlando, Fla. Vol. 1(1982), Vol. 2(1983), Vol. 3(1985); Tijssen, In: Practice and Theory of enzyme Immunoassays: Laboratory Techniques in Biochemistry and Molecular Biology, Elsevier Science Publishers, Amsterdam, The Netherlands (1985)).

The immunological assay test samples of the present invention include cells, =protein or membrane extracts of cells, or biological fluids such as blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is capable with the system utilized.

The claimed invention utilizes several suitable assays which can measure dystonia proteins. Suitable assays encompass immunological methods, such as radioimmunoassay, enzyme-linked immunosorbent assays (ELISA), and chemiluminescence assays. Any method known now or developed later can be used for performing the invention and measuring measure torsin proteins.

In several of the preferred embodiments, immunological techniques detect torsin proteins levels by means of an anti-dystonia protein antibody (i.e., one or more antibodies) which includes monoclonal and/or polyclonal antibodies, and mixtures thereof. For example, these immunological techniques can utilize mixtures of polyclonal and/or monoclonal antibodies, such as a cocktail of murine monoclonal and rabbit polyclonal.

One of skill in the art can raise anti-torsin antibodies against an appropriate immunogen, such as isolated and/or recombinant torsin proteins or a portion or fragment=thereof (including synthetic molecules, such as synthetic peptides). In one embodiment, antibodies are raised against an isolated and/or recombinant torsin proteins or a portion or fragment thereof (e.g., a peptide) or against a host cell which expresses recombinant dystonia proteins. In addition, cells expressing recombinant torsin proteins, such as transfected cells, can be used as immunogens or in a screen for antibodies which bind torsin proteins.

Any suitable technique can prepare the immunizing antigen and produce polyclonal or monoclonal antibodies. The prior art contains a variety of these methods (Kohler, et al., 1975, Nature. 256:495-497; Kohler, et al., 1976, Eur. J. Immunol. 6:511-519; Milstein, et al., 1977, Nature. 266:550-552; Koprowski, et al., U.S. Pat. No. 4,172,124; Harlow, et al., In: Antibodies: A Laboratory Manual, Cold Spring Harbor Laboratory: Cold Spring Harbor, N.Y. (1988)). Generally, fusing a suitable immortal or myeloma cell line, such as SP2/0, with antibody producing cells can produce a hybridoma Animals immunized with the antigen of interest provide the antibody-producing cell, preferably cells from the spleen or lymph nodes. Selective culture conditions isolate antibody producing hybridoma cells while limiting dilution techniques produce well established art recognized assays such as ELISA, RIA and Western blotting can be used to select antibody producing cells with the desired specificity.

Other suitable methods can produce or isolate antibodies of the requisite specificity. Examples of other methods include selecting recombinant antibody from a library or relying upon immunization of transgenic animals such as mice which are capable of producing a full repertoire of human antibodies (Jakobovits, et al., 1993, Proc. Natl. Acad. Sci. USA 90:2551-2555; Jakobovits, et al., 1993, Nature. 362:255-258; Lonbert, et al., U.S. Pat. No. 5,545,806; Surani, et al., U.S. Pat. No. 5,545,807).

According to the method, an assay can determine the level or concentration of torsin protein in a biological sample. In determining the amounts of torsin protein, an assay includes combining the sample to be tested with an antibody having specificity for torsin proteins, under conditions suitable for formation of a complex between antibody and torsin protein, and detecting or measuring (directly or indirectly) the formation of a complex. The sample can be obtained and prepared by a method suitable for the particular sample (e.g., whole blood, tissue extracts, serum) and assay format selected. For example, suitable methods for whole blood collection are venipuncture or obtaining blood from an indwelling arterial line. The container to collect the blood can contain an anti-coagulant such as CACD-A, heparin, or EDTA. Methods of combining sample and antibody, and methods of detecting complex formation are also selected to be compatible with the assay format. Suitable labels can be detected directly, such as radioactive, fluorescent or chemiluminescent labels; or indirectly detected using labels such as enzyme labels and other antigenic or specific binding partners like biotin and colloidal gold. Examples of such labels include fluorescent labels such as fluorescein, rhodamine, CY5, APC, chemiluminescent labels such as luciferase, radioisotope labels such as .sup.32p, .sup.125I, .sup.131I, enzyme labels such as horseradish peroxidase, and alkaline phosphatase, O-galactosidase, biotin, avidin, spin labels and the like. The detection of antibodies in a complex can also be done immunologically with a second antibody which is then detected. Conventional methods or other suitable methods can directly or indirectly label an antibody.

In another embodiment of the present invention, a kit is provided for diagnosing the presence or absence of a torsin protein; or the likelihood of developing a dystonia in a mammal which contains all the necessary reagents to carry out the previously described methods of detection.

For example, the kit can comprise a first container means containing an above described antibody, and a second container means containing a conjugate comprising a binding partner of the antibody and a label.

The kit can also comprise a first container means containing an above described protein, and preferably and a second container means containing a conjugate comprising a binding partner of the protein and a label. More specifically, a diagnostic kit comprises torsin protein as described above, to detect antibodies in the serum of potentially infected animals or humans.

In another preferred embodiment, the kit further comprises one or more other containers comprising one or more of the following: wash reagents and reagents capable of detecting the presence of bound antibodies. Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit can be as described above for nucleic acid probe kits. The kit can be, for example, a RIA kit or an ELISA kit.

One skilled in the art will readily recognize that the antibodies described in the present invention can readily be incorporated into one of the established kit formats which are well known in the art.

It is to be understood that although the following discussion is specifically directed to human patients, the teachings are also applicable to any animal that expresses a torsin protein. The term “mammalian,” as defined herein, refers to any vertebrate animal, including monotremes, marsupials and placental, that suckle their young and either give birth to living young (eutherian or placental mammals) or are egg-laying (metatherian or non-placental mammals). Examples of mammalian species include primates (e.g., humans, monkeys, chimpanzees, baboons), rodents (e.g., rats, mice, guinea pigs, hamsters) and ruminants (e.g., cows, horses).

The diagnostic and screening methods of the present invention encompass detecting the presence, or absence of, a mutation in a gene wherein the mutation in the gene results in a neuronal disease in a human. For example, the diagnostic and screening methods of the present invention are especially useful for diagnosing the presence or absence of a mutation or polymorphism in a neuronal gene in a human patient, suspected of being at risk for developing a disease associated with an altered expression level of torsin based on family history, or a patient in which it is desired to diagnose a torsin-related disease.

Preferably, nucleic acid diagnosis is used as a means of differential diagnosis of various forms of a torsion dystonia such as early-onset generalized dystonia; late-onset generalized dystonia; or any form of genetic, environmental, primary or secondary dystonia. This information is then used in genetic counseling and in classifying patients with respect to individualized therapeutic strategies.

According to the invention, presymptomatic screening of an individual in need of such screening is now possible using DNA encoding the torsin protein of the invention. The screening method of the invention allows a presymptomatic diagnosis, including prenatal diagnosis, of the presence of a missing or aberrant torsin gene in individuals, and thus an opinion concerning the likelihood that such individual would develop or has developed a torsin-associated disease. This is especially valuable for the identification of carriers of altered or missing torsin genes, for example, from individuals with a family history of a torsin-associated disease. Early diagnosis is also desired to maximize appropriate timely intervention.

Identification of gene carriers prior to onset of symptoms allows evaluation of genetic and environmental factors that trigger onset of symptoms. Modifying genetic factors could include polymorphic variations in torsin proteins (specifically, torsin proteins) or mutations in related or associated proteins; environmental factors include sensory overload to the part of body subserved by susceptible neurons, such as that caused by overuse or trauma (Gasser, T., et al., 1996, Mov Disord. 11:163-166); high body temperature; or exposure to toxic agents.

In one embodiment of the diagnostic method of screening, a test sample comprising a bodily fluid (e.g., blood, saliva, amniotic fluid) or a tissue (e.g., neuronal, chorionic villous) sample would be taken from such individual and screened for (1) the presence or absence of the “normal” torsin gene; (2) the presence or absence of torsin mRNA and/or (3) the presence or absence of torsin protein. The normal human gene can be characterized based upon, for example, detection of restriction digestion patterns in “normal” versus the patients DNA, including RFLP, PCR, Southern blot, Northern blot and nucleic acid sequence analysis, using DNA probes prepared against the torsin sequence (or a functional fragment thereof) taught in the invention. In one embodiment the torsin sequence is a torsin sequence (SEQ ID NOS: 1, 3, 5, 7, and 9). In another embodiment the presence or absence of three nucleotides is indicative of a negative or positive diagnosis, respectively, of a torsion dystonia Similarly, torsin mRNA can be characterized and compared to normal torsin mRNA (a) levels and/or (b) size as found in a human population not at risk of developing torsin-associated disease using similar probes. Additionally or alternatively, nucleic acids can be sequenced to determine the presence or absence of a “normal” torsin gene. Nucleic acids can be DNA (e.g., cDNA or genomic DNA) or RNA.

Lastly, torsin protein can be (a) detected and/or (b) quantitated using a biological assay for torsin activity or using an immunological assay and torsin antibodies. When assaying torsin protein, the immunological assay is preferred for its speed In one embodiment of the invention the torsin protein sequence (SEQ ID NOS: 2, 4, 6, 8, and 10) or a protein encoded by SEQ ID NOS: 1, 3, 5, 7, and 9. An (1) aberrant torsin DNA size pattern, and/or (2) aberrant torsin mRNA sizes or levels and/or (3) aberrant torsin protein levels would indicate that the patient is at risk for developing a torsin-associated disease.

Mutations associated with a dystonia disorder include any mutation in a dystonia gene, such as tor-2. The mutations can be the deletion or addition of at least one nucleotide in the coding or noncoding region, of the tor-2 gene which result in a change in a single amino acid or in a frame shift mutation.

In one method of diagnosing the presence or absence of a dystonia disorder, hybridization methods, such as Southern analysis, are used (Ausubel, et al., In: Current Protocols in Molecular Biology, John Wiley & Sons, (1998)). Test samples suitable for use in the present invention encompass any sample containing nucleic acids, either DNA or RNA. For example, a test sample of genomic DNA is obtained from a human suspected of having (or carrying a defect for) the dystonia disorder. The test sample can be from any source which contains genomic DNA, such as a bodily fluid or tissue sample. In one embodiment, the test sample of DNA is obtained from bodily fluids such as blood, saliva, semen, vaginal secretions, cerebrospinal and amniotic bodily fluid samples. In another embodiment, the test sample of DNA is obtained from tissue such as chorionic villous, neuronal, epithelial, muscular and connective tissue. DNA can be isolated from the test samples using standard, art-recognized protocols (Breakefield, X. O., et al., 1986, J. Neurogenetics. 3:159-175). The DNA sample is examined to determine whether a mutation associated with a dystonia disorder is present or absent. The presence or absence of a mutation or a polymorphism is indicated by hybridization with a neuronal gene, such as the tor-2 gene, in the genomic DNA to a nucleic acid probe. A nucleic acid probe is a nucleotide sequence of a neuronal gene. Additionally or alternatively, RNA encoded by such a probe can also be used to diagnose the presence or absence of a dystonia disorder by hybridization, a hybridization sample is formed by contacting the test sample containing a dystonia gene, such as tor-2, with a nucleic acid probe. The hybridization sample is maintained under conditions which are sufficient to allow specific hybridization of the nucleic acid probe to the dystonia gene of interest. Hybridization can be carried out as discussed previously above.

In another embodiment of the invention, deletion analysis by restriction digestion can be used to detect a deletion in a dystonia gene, such as the tor-2 gene, if the deletion in the gene results in the creation or elimination of a restriction site. For example, a test sample containing genomic DNA is obtained from the human. After digestion of the genomic DNA with an appropriate restriction enzyme, DNA fragments are separated using standard methods, and contacted with a probe specific for the a torsin gene or cDNA. The digestion pattern of the DNA fragments indicates the presence or absence of the mutation associated with a dystonia disorder. Alternatively, polymerase chain reaction (PCR) can be used to amplify the dystonia gene of interest, such as tor-2, (and, if necessary, the flanking sequences) in a test sample of genomic DNA from the human. Direct mutation analysis by restriction digestion or nucleotide sequencing is then conducted. The digestion pattern of the relevant DNA fragment indicates the presence or absence of the mutation associated with the dystonia disorder.

Allele-specific oligonucleotides can also be used to detect the presence or absence of a neuronal disease by detecting a deletion or a polymorphism associated with a particular disease by PCR amplification of a nucleic acid sample from a human with allele-specific oligonucleotide probes. An “allele-specific oligonucleotide” (also referred to herein as an “allele-specific oligonucleotide probe”) is an oligonucleotide of approximately 10-300 base pairs, that specifically hybridizes to a dystonia gene, such as tor-2, (or gene fragment) that contains a particular mutation, such as a deletion of three nucleotides. An allele-specific oligonucleotide probe that is specific for particular mutation in, for example, the tor-2 gene, can be prepared, using standard methods (Ausubel, et al., In: Current Protocols in Molecular Biology, John Wiley & Sons, (1998)).

To identify mutations in the tor-2 gene associated with torsion dystonia, or any other neuronal disease a test sample of DNA is obtained from the human. PCR can be used to amplify all or a fragment of the tor-2 gene, and its flanking sequences. PCR primers comprise any sequence of a neuronal gene. The PCR products containing the amplified neuronal gene, for example a tor-2 gene (or fragment of the gene), are separated by gel electrophoresis using standard methods (Ausubel, et al., In: Current Protocols in Molecular Biology, John Wiley & Sons, (1998)), and fragments visualized using art-recognized, well-established techniques such as fluorescent imaging when fluorescently labeled primers are used. The presence or absence of specific DNA fragments indicative of the presence or absence of a mutation or a polymorphism in a neuronal gene are then detected. For example, the presence of two alleles of a specific molecular size is indicative of the absence of a torsion dystonia; whereas the absence of one of these alleles is indicative of a torsion dystonia. The samples obtained from humans and evaluated by the methods described herein will be compared to standard samples that do and do not contain the particular mutations or polymorphism which are characteristic of the particular neuronal disorder.

Prenatal diagnosis can be performed when desired, using any known method to obtain fetal cells, including amniocentesis, chorionic villous sampling (CVS), and fetoscopy. Prenatal chromosome analysis can be used to determine if the portion of the chromosome possessing the normal torsin gene is present in a heterozygous state In the method of treating a torsin-associated disease in a patient in need of such treatment, functional torsin DNA can be provided to the cells of such patient in a manner and amount that permits the expression of the torsin protein provided by such gene, for a time and in a quantity sufficient to treat such patient. Many vector systems are known in the art to provide such delivery to human patients in need of a gene or protein missing from the cell. For example, retrovirus systems can be used, especially modified retrovirus systems and especially herpes simplex virus systems (Breakefield, X. O., et al., 1991, New Biologist. 3:203-218; Huang, Q., et al., 1992, Experimental Neurology. 115:303-316; WO93/03743; WO90/09441). Delivery of a DNA sequence encoding a functional torsin protein will effectively replace the missing or mutated torsin gene of the invention In another embodiment of this invention, the torsin gene is expressed as a recombinant gene in a cell, so that the cells can be transplanted into a mammal, preferably a human in need of gene therapy. To provide gene therapy to an individual, a genetic sequence which encodes for all or part of the torsin gene is inserted into a vector and introduced into a host cell. Examples of diseases that can be suitable for gene therapy include, but are not limited to, neurodegenerative diseases or disorders, primary dystonia (preferably, generalized dystonia and torsion dystonia).

Gene therapy methods can be used to transfer the torsin coding sequence of the invention to a patient (Chattedee and Wong, 1996, Curr. Top. Microbiol. Immunol. 218:61-73; Zhang, 1996, J. Mol. Med. 74:191-204; Schmidt-Wolf and Schmidt-Wolf, 1995, J. Hematotherapy. 4:551-561; Shaughnessy, et al., 1996, Seminars in Oncology. 23:159-171; Dunbar, 1996, Annu. Rev. Med. 47:11-20

Examples of vectors that may be used in gene therapy include, but are not limited to, defective retroviral, adenoviral, or other viral vectors (Mulligan, R. C., 1993, Science. 260:926-932). The means by which the vector carrying the gene can be introduced into the cell include but is not limited to, microinjection, electroporation, transduction, or transfection using DEAE-Dextran, lipofection, calcium phosphate or other procedures known to one skilled in the art (Sambrook, J., Fritsch, E. F., and Maniatis, T., 1989, In: Molecular Cloning. A Laboratory Manual., Cold Spring Harbor Laboratory Press, Cold Spring Harbor).

The ability of antagonists and agonists of torsin to interfere or enhance the activity of torsin can be evaluated with cells containing torsin. An assay for torsin activity in cells can be used to determine the functionality of the torsin protein in the presence of an agent which may act as antagonist or agonist, and thus, agents that interfere or enhance the activity of torsin are identified

The agents screened in the assays can be, but are not limited to, peptides, carbohydrates, vitamin derivatives, or other pharmaceutical agents. These agents can be selected and screened at random, by a rational selection or by design using, for example, protein or ligand modeling techniques (preferably, computer modeling).

For random screening, agents such as peptides, carbohydrates, pharmaceutical agents and the like are selected at random and are assayed for their ability to bind to or stimulate/block the activity of the torsin protein.

Alternatively, agents may be rationally selected or designed. As used herein, an agent is said to be “rationally selected or designed” when the agent is chosen based on the configuration of the torsin protein.

In one embodiment, the present invention relates to a method of screening for an antagonist or agonist which stimulates or blocks the activity of torsin comprising incubating a cell expressing torsin with an agent to be tested; and assaying the cell for the activity of the torsin protein by measuring the agents effect on ATP binding of torsin. Any cell may be used in the above assay so long as it expresses a functional form of torsin and the torsin activity can be measured. The preferred expression cells are eukaryotic cells or organisms. Such cells can be modified to contain DNA sequences encoding torsin using routine procedures known in the art. Alternatively, one skilled in the art can introduce mRNA encoding the torsin protein directly into the cell.

In another embodiment, the present invention relates to a screen for pharmaceuticals (e.g., drugs) which can counteract the expression of a mutant torsin protein. Preferably, a neuronal culture is used for the overexpression of the mutant form of torsin proteins using the vector technology described herein. Changes in neuronal morphology and protein distribution is assessed and a means of quantification is used. This bioassay is then used as a screen for drugs which can ameliorate the phenotype. Using torsin ligands (including antagonists and agonists as described above) the present invention further provides a method for modulating the activity of the torsin protein in a cell. In general, agents (antagonists and agonists) which have been identified to block or stimulate the activity of torsin can be formulated so that the agent can be contacted with a cell expressing a torsin protein in vivo. The contacting of such a cell with such an agent results in the in vivo modulation of the activity of the torsin proteins. So long as a formulation barrier or toxicity barrier does not exist, agents identified in the assays described above will be effective for in vivo use.

In another embodiment, the present invention relates to a method of administering torsin or a torsin ligand (including torsin antagonists and agonists) to an animal (preferably, a mammal (specifically, a human)) in an amount sufficient to effect an altered level of torsin in the animal. The administered torsin or torsin ligand could specifically effect torsin associated functions. Further, since torsin is expressed in brain tissue, administration of torsin or torsin ligand could be used to alter torsin levels in the brain.

One skilled in the art will appreciate that the amounts to be administered for any particular treatment protocol can readily be determined. The dosage should not be so large as to cause adverse side effects, such as unwanted cross-reactions, anaphylactic reactions, and the like. Generally, the dosage will vary with the age, condition, sex and extent of disease in the patient, counter indications, if any, and other such variables, to be adjusted by the individual physician. The dosages used in the present invention to provide immunostimulation include from about 0.1 μg to about 500 μg, which includes, 0.5, 1.0, 1.5, 2.0, 5.0, 10, 15, 20, 25, 30, 35, 40, 45, 50, 55, 60, 65, 70, 75, 80, 85, 90, 95, 100, 150, 200, 250, 300, 350, 400, and 450 μg, inclusive of all ranges and subranges there between. Such amount may be administered as a single dosage or may be administered according to a regimen, including subsequent booster doses, whereby it is effective, e.g., the compositions of the present invention can be administered one time or serially over the course of a period of days, weeks, months and/or years.

Also, the dosage form such as injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like can be used appropriately depending on the administration method, and the peptide of the present invention can be accordingly formulated. Pharmaceutical formulations are generally known in the art, and are described, for example, in Chapter 25.2 of Comprehensive Medicinal Chemistry, Volume 5, Editor Hansch et al, Pergamon Press 1990.

Torsin or torsin ligand can be administered parenterally by injection or by gradual perfusion over time. It can be administered intravenously, intraperitoneally, intramuscularly, or subcutaneously.

Preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media Parenteral vehicles include sodium chloride solution, Ringer's dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present, such as, for example, antimicrobials, antioxidants, chelating agents, inert gases and the like (Remington's Pharmaceutical Science, 16th ed., Eds.: Osol, A., Ed., Mack, Easton Pa. (1980)).

In another embodiment, the present invention relates to a pharmaceutical composition comprising torsin or torsin ligand in an amount sufficient to alter is torsin associated activity, and a pharmaceutically acceptable diluent, carrier, or excipient. Appropriate concentrations and dosage unit sizes can be readily determined by one skilled in the art as described above (Remington's Pharmaceutical Sciences, 16th ed., Eds.: Osol, A., Ed., Mack, EastonPA (1980); WO 91/19008).

The pharmaceutically acceptable carrier which can be used in the present invention includes, but is not limited to, an excipient, a binder, a lubricant, a colorant, a disintegrant, a buffer, an isotonic agent, a preservative, an anesthetic, and the like which are commonly used in a medical field.

The non-human animals of the invention comprise any animal having a transgenic interruption or alteration of the endogenous gene(s) (knock-out animals) and/or into the genome of which has been introduced one or more transgenes that direct the expression of human torsin.

Such non-human animals include vertebrates such as rodents, non-human primates, sheep, dog, cow, amphibians, reptiles, etc. Preferred non-human animals are selected from non-human mammalian species of animals, most preferably, animals from the rodent family including rats and mice, most preferably mice.

The transgenic animals of the invention are animals into which has been introduced by nonnatural means (i.e., by human manipulation), one or more genes that do not occur naturally in the animal, e.g., foreign genes, genetically engineered endogenous genes, etc. The non-naturally introduced genes, known as transgenes, may be from the same or a different species as the animal but not naturally found in the animal in the configuration and/or at the chromosomal locus conferred by the transgene.

Transgenes may comprise foreign DNA sequences, i.e., sequences not normally found in the genome of the host animal. Alternatively or additionally, transgenes may comprise endogenous DNA sequences that are abnormal in that they have been rearranged or mutated in vitro in order to alter the normal in vivo pattern of expression of the gene, or to alter or eliminate the biological activity of an endogenous gene product encoded by the gene (Watson, J. D., et al., In: Recombinant DNA, 2d Ed., W.H. Freeman & Co., New York (1992), pg. 255-272; Gordon, J. W., 1989, Intl. Rev. Cytol. 115:171-229; Jaenisch, R, 1989, Science. 240:1468-1474; Rossant, J., 1990, Neuron. 2:323-334).

The transgenic non-human animals of the invention are produced by introducing transgenes into the germline of the non-human animal. Embryonic target cells at various developmental stages are used to introduce the transgenes of the invention. Different methods are used depending on the stage of development of the embryonic target cell(s Microinjection of zygotes is the preferred method for incorporating transgenes into animal genome in the course of practicing the invention. A zygote, a fertilized ovum that has not undergone pronuclei fusion or subsequent cell division, is the preferred target cell for microinjection of transgenic DNA sequences. The murine male pronucleus reaches a size of approximately 20 micrometers in diameter, a feature which allows for the reproducible injection of 1-2 pL of a solution containing transgenic DNA sequences. The use of a zygote for introduction of transgenes has the advantage that, in most cases, the injected transgenic DNA sequences will be incorporated into the host animal's genome before the first cell division (Brinster, et al., 1985, Proc. Natl. Acad. Sci. USA 82:4438-4442). As a consequence, all cells of the resultant transgenic animals (founder animals) stably carry an incorporated transgene at a particular genetic locus, referred to as a transgenic allele. The transgenic allele demonstrates Mendelian inheritance: half of the offspring resulting from the cross of a transgenic animal with a non-transgenic animal will inherit the transgenic allele, in accordance with Mendel's rules of random assortment.

Viral integration can also be used to introduce the transgenes of the invention into an animal. The developing embryos are cultured in vitro to the developmental stage known as a blastocyst. At this time, the blastomeres may be infected with appropriate retroviruses (Jaenisch, R., 1976, Proc. Natl. Acad. Sci. USA 73:1260-1264). Infection of the blastomeres is enhanced by enzymatic removal of the zona pellucida (Hogan, et al., In: Manipulating the Mouse Embryo, Cold Spring Harbor Press, Cold Spring Harbor, N.Y. (1986)). Transgenes are introduced via viral vectors which are typically replication-defective but which remain competent for integration of viral-associated DNA sequences, including transgenic DNA sequences linked to such viral sequences, into the host animal's genome (Jahner, et al., 1985, Proc. Natl. Acad. Sci. USA 82:6927-6931; van der Putten, et al., 1985, Proc. Natl. Acad. Sci. USA 82:6148-6152). Transfection is easily and efficiently obtained by culture of blastomeres on a mono-layer of cells producing the transgene-containing viral vector (van derPutten, et al., 1985, Proc. Natl. Acad. Sci. USA 82:6148-6152; Stewart, et al., 1987, EMBO J. 6:383-388). Alternatively, infection may be performed at a later stage, such as a blastocoele (Jahner, D., et al., 1982, Nature. 298:623-628). In any event, most transgenic founder animals produced by viral integration will be mosaics for the transgenic allele; that is, the transgene is incorporated into only a subset of all the cells that form the transgenic founder animal. Moreover, multiple viral integration events may occur in a single founder animal, generating multiple transgenic alleles which will segregate in future generations of offspring. Introduction of transgenes into germline cells by this method is possible but probably occurs at a low frequency (Jahner, D., et al., 1982, Nature. 298:623-628). However, once a transgene has been introduced into germline cells by this method, offspring may be produced in which the transgenic allele is present in all of the animal's cells, i.e., in both somatic and germline cells.

Embryonic stem (ES) cells can also serve as target cells for introduction of the transgenes of the invention into animals. ES cells are obtained from pre-implantation embryos that are cultured in vitro (Evans, M. J., et al., 1981, Nature. 292:154-156; Bradley, M. O., et al., 1984, Nature. 309:255-258; Gossler, et al., 1986, Proc. Natl. Acad. Sci. USA 83:9065-9069; Robertson, E. J., et al., 1986, Nature. 322:445-448; Robertson, E. J., In: Teratocarcinomas and Embryonic Stem Cells: A Practical, Approach, Ed.: Robertson, E. J., IRL Press, Oxford (1987), pg. 71-112). ES cells, which are commercially available (from, e.g., Genome Systems, Inc., St. Louis, Mo.), can be transformed with one or more transgenes by established methods (Lovell-Badge, R. H., In: Teratocarcinomas and Embryonic Stem Cells: A Practical Approach, Ed.: Robertson, E. J., IRL Press, Oxford (1987), pg. 153-182). Transformed ES cells can be combined with an animal blastocyst, after which the ES cells colonize the embryo and contribute to the germline of the resulting animal, which is a chimera composed of cells derived from two or more animals (Jaenisch, R., 1988, Science. 240:1468-1474; Bradley, A., In: Teratocarcinomas and Embryonic Stem Cells. A Practical Approach, Ed.: Robertson, E. J., IRL Press, Oxford (1987), pg. 113-151). Again, once a transgene has been introduced into germline cells by this method, offspring may be produced in which the transgenic allele is present in all of the animal's cells, i.e., in both somatic and germline cells.

However it occurs, the initial introduction of a transgene is a non-Mendelian event. However, the transgenes of the invention may be stably integrated into germline cells and transmitted to offspring of the transgenic animal as Mendelian loci. Other transgenic techniques result in mosaic transgenic animals, in which some cells carry the transgenes and other cells do not. In mosaic transgenic animals in which germ line cells do not carry the transgenes, transmission of the transgenes to offspring does not occur. Nevertheless, mosaic transgenic animals are capable of demonstrating phenotypes associated with the transgenes.

Transgenes may be introduced into non-human animals in order to provide animal models for human diseases. Transgenes that result in such animal models include, e.g., transgenes that encode mutant gene products associated with an inborn error of metabolism in a human genetic disease and transgenes that encode a human factor required to confer susceptibility to a human pathogen (i.e., a bacterium, virus, or other pathogenic microorganism; Leder, et al., U.S. Pat. No. 5,175,383; Kindt, et al., U.S. Pat. No. 5,183,949; Small, et al., 1986, Cell. 46:13-18; Hooper, et al., 1987, Nature. 326:292-295; Stacey, et al., 1988, Nature. 332:131-136; Windle, et al., 1990, Nature. 343:665-669; Katz, et al., 1993, Cell. 74:1089-1100). Transgenically introduced mutations can give rise to null (“knock-out”) alleles in which a DNA sequence encoding a selectable and/or detectable marker is substituted for a genetic sequence normally endogenous to a non-human animal. Resultant transgenic non-human animals that are predisposed to a disease, or in which the transgene causes a disease, may be used to identify compositions that induce the disease and to evaluate the pathogenic potential of compositions known or suspected to induce the disease (Bems, A. J. M., U.S. Pat. No. 5,174,986), or to evaluate compositions which may be used to treat the disease or ameliorate the symptoms thereof (Scott, et al., WO 94/12627).

Offspring that have inherited the transgenes of the invention are distinguished from litter mates that have not inherited transgenes by analysis of genetic material from the offspring for the presence of biomolecules that comprise unique sequences corresponding to sequences of, or encoded by, the transgenes of the invention. For example, biological fluids that contain polypeptides uniquely encoded by the selectable marker of the transgenes of the invention may be immunoassayed for the presence of the polypeptides. A more simple and reliable means of identifying transgenic offspring comprises obtaining a tissue sample from an extremity of an animal, e.g., a tail, and analyzing the sample for the presence of nucleic acid sequences corresponding to the DNA sequence of a unique portion or portions of the transgenes of the invention, such as the selectable marker thereof. The presence of such nucleic acid sequences may be determined by, e.g., Southern blot analysis with DNA sequences corresponding to unique portions of the transgene, analysis of the products of PCR reactions using DNA sequences in a sample as substrates and oligonucleotides derived from the transgene's DNA sequence, etc.

In another embodiment, the present invention relates to a recombinant DNA molecule comprising an HSV-1 amplicon and at least one above-described torsin nucleic acid molecule.

Several features make HSV-1 an ideal candidate for vector development: (i) HSV-1 is essentially pantropic and can infect both dividing and non-dividing cells, such as neurons and hepatocytes; (ii) the HSV-1 genome can remain in neurons for long periods with at least some transcriptional activity; and (iii) the HSV-1 genome encodes more than 75 genes of which 38 are dispensable (nonessential) for viral replication in cell culture (Ward, P. L. and Roizinan, B., 1994, Trends Genet. 10:267-274). This offers the opportunity to replace large parts of the genome with foreign DNA, including one or more therapeutic genes of interest.

The technology to construct recombinant HSV-1 vectors was developed more than a decade ago (Mocarski, E. S., et al., 1980, Cell. 22:243-255; Post, L. E. and Reizman, B., 1981, Cell. 25:2227-2232; Roizman, B. and F. J. Jenkins, 1985, Science. 229:1208-1214). With the goal to create a prototype HSV-1/HSV-2 recombinant vaccine, the HSV-1 genome was deleted in certain domains in order to eliminate some loci responsible for neurovirulence, such as the viral thymidine kinase gene, and to create space for the insertion of a DNA fragment encoding the herpes simplex virus type 2 (HSV-2) glycoproteins D, G, and I (Meignier, B., et al., 1988, J. Inf. Dis. 158:602-614). Currently, recombinant herpes virus vectors are being evaluated in numerous protocols primarily for gene therapy of neurodegenerative diseases and brain tumors (Breakefield, X. O., et al, In: Cancer Gene Therapeutics, (1995), pp. 41-56; Glorioso, J. C., et al., “Herpes simplex virus as a gene-delivery vector for the central nervous system,” In: Viral vectors: Gene therapy and neuroscience applications, Eds.: Kaplitt, M. G. and Loewy, A. D., Academic Press, NY (1995), pp. 1-23).

The development of a second type of HSV-1 vector, the so-called HSV-1 “amplicon” vector, was based on the characterization of naturally occurring defective HSV-1 genomes (Frenkel, N., et al., 1976, J. Virol. 20:527-531). Amplicons carry three types of genetic elements: (i) prokaryotic sequences for propagation of plasmid DNA in bacteria, including an E. coli origin of DNA replication and an antibiotic resistance gene; (ii) sequences from HSV-1, including an ori and a pac signal to support replication and packaging into HSV-1 particles in mammalian cells in the presence of helper virus functions; and (iii) a transcription unit with one or more genes of interest (Ho, D. Y., 1994, Meth. Cell. Biol. 43:191-210) defective viruses and development of the amplicon system (Viral vectors: Gene therapy and neuroscience applications, Eds.: Kaplitt, M. G., and Loewy, A. D., Academic Press, NY (1995), pp. 2542).

In another embodiment, the present invention relates to the use of the above-described amplicon vectors for transfer of a torsin nucleic acid molecule into neurons HSV-1 has several biological properties that facilitate its use as a gene transfer vector into the CNS. These include: (i) a large transgene capacity (theoretically up to 150 kb), (ii) tropism for the CNS in vivo, (iii) nuclear localization in dividing as well as non-dividing cells, (iv) a large host cell range in tissue culture, (v) the availability of a panel of neuroattenuated and replication incompetent mutants, and (vi) the possibility to produce relatively high virus titers.

Another important property of the HSV-1 derived vector systems for the CNS is the ability of these virions to be transported retrogradely along axons. After fusion with the cell membrane, the virus capsid and associated tegument proteins are released into the cytoplasm. These capsids associate with the dynein complex which mediates energy dependent retrograde transport to the cell nucleus along microtubules (Topp, K S., et al, 1994, J. Neurosci. 14:318-325). Replication-incompetent, recombinant and amplicon HSV-1 vectors expressing the lacZ gene have been used to determine the localization and spread of vectors after injection. After single injections into many areas, including caudate nucleus, dentate gyrus and cerebellar cortex, the distribution of .beta.-galactosidase-positive cells was determined (Chiocca, E. A., et al., 1990, N. Biol. 2:739-746; Fink, D. J., et al., 1992, Hum. Gene Ther. 3:11-19; Huang, Q., et al., 1992, Exp. Neurol. 115:303-316; Wood, M., et al., 1994, Exp. Neurol. 130:127-140). Neurons and glia were transduced at the site of injection, and activity was also detected at distant secondary brain areas, in neurons that make afferent connections with the cells in the primary injection site. The retrograde transport to secondary sites is selective to neuroanatomic pathways, suggesting trans-synaptic travel of the virus capsids. Retrograde transport of an amplicon vector has been demonstrated after striatal injections in both the substantia nigra pars compacta and the locus coeruleus (Jin, B. K., et al., 1996, Hum. Gene Ther. 7:2015-2024). The ability of HSV-1 to travel by retrograde transport to neurons in afferent pathways suggests that the delivery of genes by these vectors can be spread beyond the original injection site to other regions of neuroanatomic importance.

The original report of amplicon-mediated gene delivery to neurons used primary cells in culture (Geller, A. I. and Breakefield, X. O. 1988, Science 241:1667-1669). Amplicon vectors have been used to study neuronal physiology, for example effects of expression of GAP43 or the low affinity nerve growth factor (NGF) receptor on morphology and growth of neuronal cells (Neve, R. L., et al., 1991, Mol. Neurobiol. 5:131-141; Battleman, D., et al., 1993, J. Neurosci. 13:941-951). Amplicons can direct rapid and stable transgene expression in hippocampal slice cultures (Bahr, B., et al., 1994, Mol. Brain Res. 26:277-285), and this has been used to model both kainate receptor-mediated toxicity (Bergold, P. J., et al., 1993, Proc. Natl. Acad. Sci. USA 90:6165-6169) and glucose transporter-mediated protection of neurons (Ho, D. Y., et al., 1995, J. Neurochem. 65:842-850). In vivo, amplicons have been used to deliver a number of candidate therapeutic genes in different models of CNS diseases. For example, expression of the glucose transporter protects neurons in an induced seizure model ((Ho, D. Y., et al., 1995, J. Neurochem. 65:842-850; Lawrence, M. S., et al., 1995, Proc. Natl. Acad. Sci. USA 92:7247-7251; Lawrence, M. S., et al., 1996, Blood Flow Metab. 16:181-185), bcl-2 rescues neurons from focal ischemia (Linnik, M. D., et al., 1995, Stroke 26:1670-1674), and expression of TH mediates behavioral changes in parkinsonian rats (During, M. J., et al., 1994, Science 266:1399-1403). Thus, amplicons have proven effective for functional expression of many transgenes in the CNS Amplicons have recently been used to generate mouse somatic mosaics, in which the expression of a host gene is activated in a spatial and developmentally regulated fashion. Transgenic mice were engineered with a germline transmitted NGF gene that contained an inactivating insertional element between the promoter and transcript flanked by the loxP sites. The somatic delivery of cre recombinase by an amplicon vector successfully activated the expression of NGF in these animals (Brooks, A. I., et al., 1997, Nat. Biotech. 15:57-62). The ability to express genes in specific cells at various points in development will have broad applications, especially for genes for which germline deletion (“knockouts”) are conditional lethal mutants.

Traditionally, the stability of transgene expression after transduction, and the cytopathic effect of the helper virus were the limiting features of amplicon mediated gene delivery into cells of the CNS. Recent advancements have largely addressed these constraints. Several promoter elements, such as preproenkephalin and tyrosine hydroxylase, can drive long-term transgene expression from amplicon vectors when upstream regulatory sequences are included (Kaplitt, M. G., et al., 1994, Proc. Natl. Acad. Sci. USA 91:8979-8983; Jin, B. K, et al., 1996, Hum. Gene Ther. 7:2015-2024). The development of hybrid amplicons containing non-HSV genetic elements that can potentially integrate in a site directed manner (Johnston, K. M., et al., 1997, Hum. Gene Ther. 8:359-370), or form stable replicating episomes (Wang, S. and Vos, J., 1996, J. Virol. 70:8422-8430), should maintain the-introduced transgene in a emetically stable configuration. Finally, the development of a packaging system devoid of contaminating helper virus (Fraefel, C., et al., 1996, J. Virol. 70:7190-7197) has significantly reduced the cytopathic effects of amplicon vectors in culture and in vivo. The easily manipulated plasmid-based amplicon, and the helper virus-free packaging system allows the construction of a virtually synthetic vector which retains the biological advantages of HSV-1, but reduces the risks associated with virus-based gene therapy.

]In another embodiment, the present invention relates to the use of the above-described amplicon vectors for transfer of a torsin nucleic acid molecule into hepatocytes. As discussed in the previous section, HSV-I amplicon vectors have been extensively evaluated for gene transfer into cells of the nervous system. However, amplicon vectors can also be an efficient means of gene delivery to other tissues, such as the liver. Certain hereditary liver disorders can be treated by enzyme/protein replacement or by liver transplantation. However, protein infusion can only temporarily restore the deficiency and is not effective for many intracellular proteins. Liver transplantation is limited by donor organ availability and the need for immunosuppression for the lifetime of the patient. Thus, gene transfer to the liver is highly desirable, and consequently, various virus vector systems, including adenovirus vectors (Stratford-Perricaudet, L. D., et al., 1990, Hum. Gene Ther. 1:241-256; Jaffe, A. H., et al., 1992, Nat. Genet. 1:372-378; L1, Q., et al., 1993, Hum. Gene Ther. 4:403-409; Herz, J. and Gerard, R. D., 1993, Proc. Natl. Acad. Sci. USA 90:2812-2816), retrovirus vectors (Hafenrichter, D. G., et al., 1994, Blood 84:3394-3404), baculovirus vectors (Boyce, F. M. and Bucher, N. R. L., 1996, Proc. Natl. Acad. Sci. USA 93:2348-2352; Sandig, V., et al., 1996, Hum. Gene Ther. 7:1937-1945) and vectors based on HSV-I (Miyanohara, A., et al., 1992, New Biologist 4:238-246; Lu, B., et al., 1995, Hepatology 21:752-759; Fong, Y., et al., 1995, Hepatology 22:723-729; Tung, C., et al., 1996, Hum. Gene Ther. 7:2217-2224) have been evaluated for gene transfer into hepatocytes in culture and in experimental animals. Recombinant HSV-1 vectors have been used to express hepatitis B virus surface antigen (HBsAG), E. coli .beta-galactosidase, and canine factor IX-CFM in infected mouse liver (Miyanohara, A., et al., 1992, New Biologist 4:238-246). Virus stocks were either injected directly into the liver parenchyma or applied via the portal vein. By either route, gene transfer proved to be highly efficient and resulted in high levels of HB SAG or CF1X in the circulation, and in a large number of .beta.-galactosidase-positive hepatocytes. Although detectable gene expression was transient, a significant number of vector genomes was demonstrated to persist for up to two months after gene transfer. The efficiency of long term gene expression could be increased somewhat by replacing the HCMV IE1 promoter with the HSV-1 LAT promoter to direct the expression of the transgene.

“protein aggregation” within the scope of the present invention includes the phenomenon of at least two polypeptides contacting each other in a manner that causes either one of the polypeptides to be in a state of de-solvation. This may also include a loss of the polypeptide's native functional activity.

“De-solvation” within the scope of the present invention is a state in which the polypeptide is not in solution.

“Treating” within the scope of the present invention reducing, inhibiting, ameliorating, or preventing. Preferably, protein aggregation, cellular dysfunction as a result of protein aggregation and protein-aggregation-associated diseases may be treated.

“Protein-aggregation-associated disease” within the scope of the present invention includes any disease, disorder, and/or affliction, protein-aggregation-associated disease include Neurodegenerative disorders.

“Neurodegenerative disorders” are Alzheimer's disease, Parkinson's disease, prion diseases, Huntington's disease, frontotemporal dementia, and motor neuron disease. They all share a conspicuous common feature: aggregation and deposition of abnormal protein (Table 1). Expression of mutant proteins in transgenic animal models recapitulates features of these diseases (A. Aguzzi and A. J. Raeber, Brain Pathol. 8, 695 (1998)). Neurons are particularly vulnerable to the toxic effects of mutant or misfolded protein. The common characteristics of these neurodegenerative disorders suggest parallel approaches to treatment, based on an understanding of the normal cellular mechanisms for disposing of unwanted and potentially noxious proteins. The following is a detailed explanation of such diseases, their cellular malfunctions, and specific examples of their respective proteins that aggregate that are known thus far.

Correct folding requires proteins to assume one particular structure from a constellation of possible but incorrect conformations. The failure of polypeptides to adopt their proper structure is a major threat to cell function and viability. Consequently, elaborate systems have evolved to protect cells from the deleterious effects of misfolded proteins. The first line of defense against misfolded protein is the molecular chaperones, which associate with nascent polypeptides as they emerge from the ribosome, promoting correct folding and preventing harmful interactions (J. P. Taylor, et al., Science 296, 1991 (2002)).

TABLE 1
Features of neurodegenerative disorders caused by protein aggregation.

| | Protein | | Disease | |
|---|---|---|---|---|
| Disease | deposits | Toxic protein | genes | Risk factor |
| Alzheimer's | Extracellular | αβ | APP | apoE4 allele |
| disease | plaques | | Presenilin 1 | |
| | | | Presenilin 2 | |
| | Intracellular | tau | | |
| | tangles | | | |
| Parkinson's | Lewy bodies | alpha- | alpha- | tau linkage |
| disease | | Synuclein | Synuclein | |
| | | | Parkin | |
| | | | UCHL1 | |
| Prion disease | Prion plaque | PrP^{Sc} | PRNP | Homozygosity |
| | | | | at prion codon 129 |
| Polyglutamine | Nuclear and | Polyglutamine- | 9 different | |
| disease | cytoplasmic | containing | genes with | |
| | inclusions | proteins | CAG repeat | |
| | | | expansion | |
| Tauopathy | Cytoplasmic | tau | tau | tau linkage |
| Familial | tangles Bunina | SOD1 | SOD1 | |
| amyotrophic | bodies | | | |
| lateral sclerosis | | | | |

Alzheimer's disease is the most common neurodegenerative disease, directly affecting about 2 million Americans. It is characterized by the presence of two lesions: the plaque, an extracellular lesion made up largely of the β-amyloid (A) peptide, and the tangle, an intracellular lesion made up largely of the cytoskeletal protein tau. Although it is predominantly a disease of late life, there are families in which Alzheimer's disease is inherited as an autosomal dominant disorder of midlife. Three genes have been implicated in this form of the disease: the amyloid precursor protein (APP) gene (A. M. Goate, et al., Nature 349, 704 (1991)), which encodes the A peptide; and the presenilin protein genes (PS1 and PS2), which encode transmembrane proteins (R. Sherrington, et al., Nature 375, 754 (1995); E. Levy-Lahad, et al., Science 269, 973 (1995)).

Metabolism of APP generates a variety of A species, predominantly a 40-amino acid peptide, A1-40, with a smaller amount of a 42-amino acid peptide, A1-42. This latter form of the peptide is more prone to forming amyloid deposits. Mutations in all three pathogenic genes alter the processing of APP such that a more amyloidogenic species of A is produced (D. Scheuner, et al., Nature Med. 2, 864 (1996)). Although the precise function of the presenilins is still the subject of debate, it is clear from gene ablation experiments that presenilins are intimately involved in the COOH-terminal cleavage of A (B. De Strooper, et al., Nature 391, 387 (1998)), and the simplest explanation of the effects of presenilin mutations on APP processing is that they lead to an incomplete loss of function of the complex that processes APP (L. M. Refolo, et al., J. Neurochem. 73, 2383 (1999); M. S. Wolfe et al., Nature 398, 513 (1999)).

The implication of these findings is that the process of A deposition is intimately connected to the initiation of Alzheimer pathogenesis and that all the other features of the disease, i.e. the tangles and the cell and synapse loss, are secondary to this initiation; this is the amyloid cascade hypothesis for Alzheimer's disease (J. A. Hardy and G. A. Higgins, Science 286, 184 (1992)). If this hypothesis is correct, then other genetic or environmental factors that promote A deposition are likely to predispose to the disease, and seeking treatments that prevent this deposition is a rational route to therapy. The only gene confirmed to confer increased risk for typical, late-onset Alzheimer's disease is the apolipoprotein E4 allele (E. H. Corder, et al., Science 261, 921 (1993)), and apolipoprotein E gene knockouts have been shown to prevent A deposition (K. R. Bales, et al., Proc. Natl. Acad. Sci. U.S.A. 96, 15233 (1999)), consistent with the amyloid cascade hypothesis. Other genes predisposing to Alzheimer's disease are being sought, and it seems most likely that they too act by alteration of A metabolism (A. Myers, et al., Science 290, 2304 (2000); N. Ertekin-Taner, et al., Science 290, 303 (2000)).

These findings suggest that A metabolism is the key pathway to be targeted for therapy, and there has been much progress in this arena with transgenic mice that develop plaque pathology (D. Schenk, et al., Nature 400, 173 (1999)). Immunization of these transgenic mice with A results in a reduction in pathology and better performance in behavioral tests, providing evidence that A-directed therapy may be clinically relevant (D. Morgan, et al., Nature 408, 982 (2000)). Immunization may not turn out to be a practical approach to therapy, but the results of these animal studies have been an important proof of principle. It should be noted, however, that the APP transgenic mice used in these studies do not show tangles or cell loss, and it will be important to retest this strategy in newer, more complete models of the disease (J. Lewis, et al., Science 293, 1487 (2001)).

Parkinson's disease affects about half a million individuals in the United States and previously has been considered a nongenetic disorder. However, recent data increasingly implicate genetic factors in its etiology. Two genes are clearly associated with the disease: α-synuclein (PARK1) (M. H. Polymeropoulos, et al., Science 276, 2045 (1997)) and parkin PARK2) (T. Kitada, et al., Nature 392, 605 (1998)). There is evidence implicating a third, ubiquitin COOH-terminal hydrolase (PARK5) (E. Leroy, et al., Nature 395, 451 (1998); D. M. Maraganore, et al., Neurology 53, 1858 (1999)), and there are at least five other linkage loci (PARK 3, 4, 6, 7, and 8), indicating additional contributing genes (M. Farrer, et al., Hum. Mol. Genet. 8, 81 (1999); . T. Gasser, et al., Nature Genet. 18, 262 (1998); E. M. Valente, et al., Am. J. Hum. Genet. 68, 895 (2001); C. M. Van Duijn, et al., Am. J. Hum. Genet. 69, 629 (2001); A. Hicks et al., Am. J. Hum. Genet. 69 (suppl.), 200 (2001); M. Funayama, et al., Ann. Neurol. 51, 296 (2002)). The pathological hallmark of Parkinson's disease is the deposition within dopaminergic neurons of Lewy bodies, cytoplasmic inclusions composed largely of α-synuclein. As the work on Alzheimer's disease has suggested, when multiple genes influence a single disorder, those genes may define a pathogenic biochemical pathway. It is not yet clear what this pathway might be in Parkinson's disease. The notion that it could be a pathway involved in protein degradation (E. Leroy, et al., Nature 395, 451 (1998)) has gained ground with the observations that parkin is a ubiquitin-protein ligase (H. Shimura, et al., Nature Genet. 25, 302 (2001)) and that parkin and α-synuclein may interact (H. Shimura, et al., Science 293, 263 (2001)). In at least one patient, mutations in parkin led to Lewy body formation as seen in sporadic Parkinson's disease (M. Farrer, et al., Ann. Neurol. 50, 293 (2001)). The interaction of parkin with α-synuclein may be mediated by synphilin-1 (K. K. Chung, et al., Nature Med. 7, 1144 (2001)). Another pathologically relevant substrate for parkin is the unfolded form of Pael, which is found to accumulate in the brains of patients with parkin mutations (Y. Imai, et al., Cell 105, 891 (2001)). If protein degradation is the key pathogenic pathway in Parkinson's disease, one may predict that additional Parkinson's disease loci encode other proteins in this same pathway. Dopaminergic neurons may be more sensitive to the disease process than other neurons because they sustain more protein damage through oxidative stress induced by dopamine metabolism. However, work on the molecular basis of Parkinson's disease is currently less advanced than work on other neurodegenerative diseases; as additional genes are found, other pathogenic mechanisms may emerge.

The most common human prion disease is sporadic Creutzfeldt-Jacob disease (CJD). Less common are the hereditary forms, including familial CJD, Gerstmann-Straussler-Scheinker disease, and fatal familial insomnia (S. B. Prusiner, N. Engl. J. Med. 344, 1516 (2001)). Prion diseases are distinct from other neurodegenerative disorders by virtue of their transmissibility. Although they share a common molecular etiology, the prion diseases vary greatly in their clinical manifestations, which may include dementia, psychiatric disturbance, disordered movement, ataxia, and insomnia The pathology of prion diseases shows varying degrees of spongioform vacuolation, gliosis, and neuronal loss. The one consistent pathological feature of the prion diseases is the accumulation of amyloid material that is immunopositive for prion protein (PrP), which is encoded by a single gene on the short arm of chromosome 20.

Substantial evidence now supports the contention that prions consist of an abnormal isoform of PrP (J. Collinge, Annu. Rev. Neurosci. 24, 519 (2001)). Structural analysis indicates that normal cellular PrP (designated PrPC) is a soluble protein rich in α-helix with little β-pleated sheet content. In contrast, PrP extracted from the brains of affected individuals (designated PrPSc) is highly aggregated and detergent insoluble. PrPSc is less rich in helix and has a greater content of β-pleated sheet. The polypeptide chains for PrPC and PrPSc are identical in amino acid composition, differing only in their three-dimensional conformation.

It is suggested that the PrP fluctuates between a native state (PrPC) and a series of additional conformations, one or a set of which may self-associate to produce a stable supramolecular structure composed of misfolded PrP monomers (J. Collinge, Annu. Rev. Neurosci. 24, 519 (2001)). Thus, PrPSc may serve as a template that promotes the conversion of PrPC to PrPSc. Initiation of a pathogenic self-propagating conversion reaction may be induced by exposure to a “seed” of β-sheet-rich PrP after prion inoculation, thus accounting for transmissibility. The conversion reaction may also depend on an additional, species-specific factor termed “protein X” (K. Kaneko, et al., Proc. Natl. Acad. Sci. U.S.A. 94, 10069 (1997)). Alternatively, aggregation and deposition of PrPSc may be a consequence of a rare, stochastic conformational change leading to sporadic cases. Hereditary prion disease is likely a consequence of a pathogenic mutation that predisposes PrPC to the PrPSc structure.

At least nine inherited neurological disorders are caused by trinucleotide (CAG) repeat expansion, including Huntington's disease, Kennedy's disease, dentatorubro-pallidoluysian atrophy, and six forms of spinocerebellar ataxia (H. Y. Zoghbi and H. T. Orr, Annu. Rev. Neurosci. 23, 217 (2000); K. Nakamura, et al., Hum. Mol. Genet. 10, 1441 (2001)). These are all adult-onset diseases with progressive degeneration of the nervous system that is typically fatal. The genes responsible for these diseases appear to be functionally unrelated. The only known common feature is a CAG trinucleotide repeat in each gene's coding region, resulting in a polyglutamine tract in the disease protein. In the normal population, the length of the polyglutamine tract is polymorphic, generally ranging from about 10 to 36 consecutive glutamine residues. In each of these diseases, however, expansion of the polyglutamine tract beyond the normal range results in adult-onset, slowly progressive neurodegeneration. Longer expansions correlate with earlier onset, more severe disease.

These diseases likely share a common molecular pathogenesis resulting from toxicity associated with the expanded polyglutamine tract. It is now clear that expanded polyglutamine endows the disease proteins with a dominant gain of function that is toxic to neurons. Each of the polyglutamine diseases is characterized by a different pattern of neurodegeneration and thus different clinical manifestations. The selective vulnerability of different populations of neurons in these diseases is poorly understood but likely is related to the expression pattern of each disease gene and the normal function and interactions of the disease gene product. Partial loss of function of individual disease genes, although not sufficient to cause disease, may contribute to selective neuronal vulnerability (. I. Dragatsis, M. S. Levine, S. Zeitlin, Nature Genet. 26, 300 (2000); C. Zuccato et al. Science 293, 493 (2001)).

Several years ago, it was recognized that expanded polyglutamine forms neuronal intranuclear inclusions in animal models of the polyglutamine diseases and the central nervous system of patients with these diseases (C. A. Ross, Neuron 19, 1147 (1997)). These inclusions consist of accumulations of insoluble aggregated polyglutamine-containing fragments in association with other proteins. It has been proposed that proteins with long polyglutamine tracts misfold and aggregate as antiparallel strands termed “polar zippers” (M. F. Perutz, Proc. Natl. Acad. Sci. U.S.A. 91, 5355 (1994)). The correlation between the threshold polyglutamine length for aggregation in experimental systems and the CAG repeat length that leads to human disease supports the argument that self-association or aggregation of expanded polyglutamine underlies the toxic gain of function. Although in some experimental systems the toxicity of expanded polyglutamine has been dissociated from the formation of visible inclusions, the formation of insoluble molecular aggregates appears to be a consistent feature of toxicity (. S. Sisodia, Cell 95, 1 (1998); I. A. Klement, et al., Cell 95, 41 (1998); F. Saudou, S. Finkbeiner, D. Devys, M. E. Greenberg, Cell 95, 55 (1998); P. J. Muchowski, et al., Proc. Natl. Acad. Sci. U.S.A. 99, 727 (2002)). The observed correlation between aggregation and toxicity in the polyglutamine diseases suggests a link with the other neurodegenerative diseases characterized by deposition of abnormal protein.

Tau has long been suspected of playing a causative role in human neurodegenerative disease, a view supported by the observation that filamentous tau inclusions are the predominant neuropathological feature of a broad range of sporadic disorders, including Pick's disease, corticobasal degeneration (CBD), progressive supranuclear palsy (PSP), and the amyotrophic lateral sclerosis/parkinsonism-dementia complex. This group of disorders is collectively referred to as the “tauopathies” (V. M-Y. Lee, M. Goedert, J. Q. Trojanowski, Annu. Rev. Neurosci. 24, 1121 (2001)). Filamentous tau deposition is also frequently observed in the brains of patients with Alzheimer's disease and prion diseases. The tau proteins are low molecular weight, microtubule-associated proteins that are abundant in axons of the central and peripheral nervous system. Encoded by a single gene on chromosome 17, multiple tau isoforms are generated by alternative splicing. The discovery that multiple mutations in the gene encoding tau are associated with frontotemporal dementia and parkinsonism (FTDP-17) provided strong evidence that abnormal forms of tau may contribute to neurodegenerative disease (L. A. Reed, Z. K Wszolek, M. Hutton, Neurobiol. Aging 22, 89 (2001)). Moreover, polymorphisms associated with the tau gene appear to be risk factors for sporadic CBD, PSP, and Parkinson's disease (E. R. Martin, et al., J. Am. Med. Assoc. 286, 2245 (2001); N. Cole and T. Siddique, Semin. Neurol. 19, 407 (1999)). Emerging evidence suggests that tau abnormalities associated with neurodegenerative disease impair tau splicing, favor fibrillization, and generally promote the deposition of tau aggregates.

Amyotrophic lateral sclerosis (ALS) is a progressive neurodegenerative disease of upper and lower motor neurons. About 10% of ALS cases are inherited; the remainder are believed to be sporadic cases (N. Cole and T. Siddique, Semin. Neurol. 19,407 (1999)). Of the inherited cases, about 20% are caused by mutations in the gene encoding superoxide dismutase 1 (SOD1). More than 70 different pathogenic SOD1 mutations have been described; all are dominant except for the substitution of valine for alanine at position 90, which may be recessive or dominant. Neuropathologically, ALS is characterized by degeneration and loss of motor neurons and gliosis. Intracellular inclusions are found in degenerating neurons and glia (L. P. Rowland and N. A. Shneider, N. Engl. J. Med. 344, 1688 (2001)). Familial ALS is characterized neuropathologically by neuronal Lewy body-like hyaline inclusions and astrocytic hyaline inclusions composed largely of mutant SOD1.

SOD1 is a copper-dependent enzyme that catalyzes the conversion of toxic superoxide radicals to hydrogen peroxide and oxygen. Mutations that impair the antioxidant function of SOD1 could lead to toxic accumulation of superoxide radicals. However, a loss-of-function mechanism for familial ALS is unlikely given that no motor neuron degeneration is seen in transgenic mice in which SOD1 expression has been eliminated. Moreover, overexpression of mutant SOD1 in transgenic mice causes motor neuron disease despite elevated SOD1 activity. This supports a role for a deleterious gain of function by the mutant protein, consistent with autosomal dominant inheritance. A pro-oxidant role for mutant SOD1 contributing to motor neuron degeneration has been proposed. This seems unlikely, however, given that ablation of the specific copper chaperone for SOD1, which deprives SOD1 of copper and eliminates enzymatic activity, has no effect on motor neuron degeneration in mutant SOD1 transgenic mice (J. R Subrananiam, et al., Nature Neurosci. 5, 301 (2002)). More recently, attention has turned to the possible deleterious effects of accumulating aggregates of mutant SOD1. The notion that aggregation is related to pathogenesis is supported by the observation that murine models of mutant SOD1-mediated disease feature prominent intracellular inclusions in motor neurons, and in some cases within the astrocytes surrounding them as well (D. W. Cleveland and J. Liu, Nature Med. 6, 1320 (2000)). Although a variety of inclusions have been described in sporadic cases of ALS, there is scant evidence for deposition of SOD1 in these inclusions and no convincing evidence that aggregation contributes to the pathogenesis of sporadic ALS.

It remains unclear exactly how abnormal proteins could lead to neurodegenerative disease. Determining the mechanism of toxicity of mutant or misfolded, aggregation-prone protein remains the most important unresolved research problem for each of these diseases. Although the different diseases may ultimately involve different mechanisms, certain common themes have emerged, which could point the way to common therapeutic approaches.

Proposed mechanisms of toxicity include sequestration of critical factors by the abnormal protein (A. McCampbell and K. H. Fischbeck, Nature Med. 7, 528 (2001); J. S. Steffan, et al., Proc. Natl. Acad. Sci. U.S.A. 97, 6763 (2000); F. C. Nucifora, et al., Science 291, 2423 (2001)), inhibition of the UPS (4), inappropriate induction of caspases and apoptosis (M. P. Mattson, Nature Rev. Mol. Cell Biol. 1, 120 (2000)), and inhibition by aggregates of neuron-specific functions such as axonal transport and maintenance of synaptic integrity (D. W. Cleveland, Neuron 24, 515 (1999); P. F. Chapman, et al., Nature Neurosci. 2, 271 (1999)). For example, mutant polyglutamine-containing proteins bind and deplete CREB-binding protein and other protein acetylases (A. McCampbell and K H. Fischbeck, Nature Med. 7, 528 (2001); J. S. Steffan, et al., Proc. Natl. Acad. Sci. U.S.A. 97, 6763 (2000); F. C. Nucifora, et al., Science 291, 2423 (2001)). That this may contribute to polyglutamine toxicity is supported by the finding that deacetylase inhibitors can mitigate the toxic effect (A. McCampbell, et al., Proc. Natl. Acad. Sci. U.S.A. 98, 15179 (2001); J. S. Steffan, et al., Nature 413, 739 (2001)). There is recent evidence that mutant polyglutamine can impede proteasome activity (N. F. Bence, R. M. Sampat, R. R. Kopito, Science 292, 1552 (2001)); the key role of proteasomes in maintaining cell viability indicates that this effect of the mutant protein could be important in mediating neuronal dysfunction and death. Caspase activation and apoptosis have been well demonstrated in cell culture models of polyglutamine disease, ALS, and Alzheimer's disease (M. P. Mattson, Nature Rev. Mol. Cell Biol. 1, 120 (2000)), and the role of apoptosis in polyglutamine disease and ALS is indicated by the mitigating effects of caspase inhibition in transgenic mouse models (D. W. Cleveland, Neuron 24, 515 (1999)). Demonstration of apoptosis in patient autopsy samples is more difficult, perhaps because of the long time course and slow evolution of these disorders in humans or because different cell death pathways may be involved (S. Sperandio, L de Belle, D. E. Bredesen, Proc. Natl. Acad. Sci. U.S.A. 97, 14376 (2000)). Neurofilament changes and defects in axonal transport occur in ALS (D. W. Cleveland, Neuron 24, 515 (1999)), and early synaptic pathology has been found in transgenic models of Alzheimer's disease (P. F. Chapman, et al., Nature Neurosci. 2, 271 (1999)). Other implicated mechanisms include excitotoxicity, mitochondrial dysfunction, oxidative stress, and the microglial inflammatory response. Indeed, downstream from the direct effects of mutant or misfolded protein in neurodegenerative diseases the mechanisms of toxicity likely diverge.

These insights into the role of toxic proteins in neurodegenerative disease suggest rational approaches to treatment. First, blocking the expression or accelerating the degradation of the toxic protein can be an effective therapy. Reducing expression of the mutant polyglutamine in transgenic mice can reverse the phenotype (A. Yamamoto, J. J. Lucas, R. Hen, Cell 101, 57 (2000)), and immune-mediated clearance of β-amyloid has a similar benefit in an animal model of Alzheimer's disease (D. Morgan, et al., Nature 408, 982 (2000)). Because fragments of the toxic proteins may be more pathogenic than the full-length protein and specific cellular localization may enhance toxicity, blocking proteolytic processing and intracellular transport are reasonable approaches to treatment. Other therapeutic strategies include inhibiting the tendency of the protein to aggregate (either with itself or with other proteins), up-regulating heat shock proteins that protect against the toxic effects of misfolded protein, and blocking downstream effects, such as triggers of neuronal apoptosis. Overexpression of heat shock protein can reduce the toxicity of both mutant polyglutamine and mutant α-synuclein (J. M. Warrick, et al., Nature Genet. 23, 425 (1999); P. K. Auluck, et al., Science 295, 865 (2002)), and caspase inhibition can reduce the toxicity of both polyglutamine and mutant SOD (V. O. Ona, et al., Nature 399, 263 (1999); M. W. L1, et al., Science 288, 335 (2000)), indicating that therapeutic interventions of this type may apply across multiple neurodegenerative diseases. Pharmaceutical screens are now under way to identify agents that block the expression or alter the processing and aggregation of the toxic proteins responsible for neurodegenerative disease, or mitigate the harmful effects of these proteins on neuronal function and survival.

The molecular basis for torsion dystonia remains unclear. Ozelius et al. identified the causative gene, named TOR1A, and mapped it to human chromosome 9q34 (L. J. Ozelius, et al., Nature Genetics 17, 40 (1997)). The TOR1A gene produces a protein named TOR-A. The majority of patients with early onset torsion dystonia have a unique deletion of one codon, which results in a loss of glutamic acid (GAG) residue at the carboxy terminal of TOR-A. A misfunctional torsin protein is produced Notably, this was the only change observed on the disease chromosome (L. J. Ozelius, et al., Genomics 62, 377 (1999); L. J. Ozelius, et al., Nature Genetics 17, 40 (1997)). A recent paper described an additional deletion of 18 base pairs or 6 amino acids at the carboxy terminus. This is the first mutation identified beyond the GAG deletion (L. J. Ozelius, et al., Nature Genetics 17, 40 (1997)).

In Caenorhabditis elegans, the homolog with highest amino acid sequence identity to the human TOR1A gene is the tor-2 gene product. This nematode also contains a second torsin gene named tor-1. In the original paper identifying the TOR1A gene, a nematode torsin-like protein was described, which has since been shown to encode the ooc-5 gene (L. J. Ozelius, et al., Nature Genetics 17,40 (1997), S. E. Basham, and L. E. Rose, Dev Biol 215 253 (1999)). The three Celegans torsin genes share a high sequence identity to each other (L. J. Ozelius, et al., Nature Genetics 17, 40 (1997)).

The genes tor-1 and tor-2 are situated next to each other on chromosome IV of C elegans and are oriented in the same direction. These two genes are separated by only 348 base pairs. This implies that perhaps these genes are positioned together to form an operon unit (Blumenthal, T. 1998. Gene clusters and polycistronic transcription in eukaryotes. Bioessays 6: 480-487). Interestingly, humans also have two torsin genes, TOR1A and TOR1B, that produce the proteins torsin A and torsin B. These two proteins have a 70% sequence similarity (L. J. Ozelius, et al., Genomics 62, 377 (1999)). The human genes also lie on the same chromosome (9q34), but in opposite directions (L. J. Ozelius, et al., Nature Genetics 17, 40 (1997); Ozelius L J, Hewett J W, Page C E, Bressman S B, Kramer P L, Shalish C, de Leon D, Brin M F, Raymond D, Jacoby D, Penney J, Fahn S, Gusella J F, Risch N J, Breakefield X O. 1998. The gene (DYT1) for early-onset torsion dystonia encodes a novel protein related to the Clp protease/heat shock family. Advances in Neurology. 78:93-105).

The TOR-A protein shares a distant similarity (25%-30%) to the AAA+/Hsp 100/Clp family of proteins (L. J. Ozelius, et al., Genomics 62, 377 (1999); Neuwald AF, Aravind L, Spouge J L, Koonin E V. 1999. AAA+: A class of chaperone-like ATPases associated with the assembly, operation, and disassembly of protein complexes. Genome Res 9: 2743). Members of this family are ATPases of diverse function, hinder protein aggregation by binding to exposed surfaces, and regulate the repair of damaged substrates (Schirmer E C, glover J R, Singer M A, Lindquist S. 1996. Hsp 100/Clp proteins: a common mechanism explains diverse functions. Trends Biochem Sci 21:289-296) Heat shock proteins have several different activities related to chaperone functions. They prevent misfolding of proteins, regulate protein signaling, and allow for the correct localization of the proteins. Heat shock proteins are thought to be activated when other proteins in a cell do not fold correctly. If heat shock protein activation fails, misfolded proteins tend to form aggregates. This could represent a possible cause of diseases such as Alzheimer's, Parkinson's and Huntington's wherein protein aggregates form. Recently, it has been shown that the Hsp 40 and the Hsp 70 heat shock families are involved in preventing polyglutamine aggregation (Chai Y, Koppenhafer S L, Bonini N M, and Paulson H L. 1999. Analysis of the Role of Heat Shock Protein (Hsp) Molecular Chaperones in Polyglutamine Disease. The Journal of Neuroscience. 19(23):10338-10347) In examining the polyglutamine neurodegenerative disease spinocerebellar ataxia 3, also called Machado-Joseph Disease, and its associated disease-causing protein ataxin 3, they studied the consequences of aggregates on the cells and the effects of chaperones on the polyglutamine aggregates. Their experiments showed that Hsp 40 and Hsp 70 are used as part of the cell's response to polyglutamine aggregates. These chaperones are able to diminish the toxic effects of the aggregates. The presence of the mutant ataxin-3 induced a stress response in the cells and activated the chaperone Hsp 70. Thus, the cell views the polyglutamine protein as abnormal and recruits its chaperones to aid in suppression of these aggregates.

Further implying that perhaps torsin proteins have a chaperone function was the recent finding that torsin A is localized to intracellular membranes (Kustedjo K, Bracey M H, Cravatt B F. Torsin A and Its Torsin Dystonia-associated Mutant Forms Are Lumenal Glycoproteins That Exhibit Distinct Subcellular Localizations. 2000. J of Biol Chem 275:27933-27939). Using immunofluroescence, TOR-A was shown to have high co-localization with the ER resident protein, BiP. Interestingly, the mutant form of TOR-A, lacking a glutamic acid residue as found in dystonia patients, was located in large aggregate-like formations absent of BiP immunoreactivity (Kustedjo K, Bracey M H, Cravatt B F. Torsin A and Its Torsin Dystonia-associated Mutant Forms Are Lumenal Glycoproteins That Exhibit Distinct Subcellular Localizations. 2000. J of Biol Chem 275:27933-27939). This supports another report that torsin A is glycosylated, a characteristic of ER proteins, and is co-localized with PDL an ER marker. Mutant TOR-A was also shown to develop large cytoplasmic inclusions (Hewett J, Gonzalez-Agosti C, Slater D, Ziefer P, Li S, Bergeron D, Jacoby D J, Ozelius L J, Ramesh V, and Breakefield X O. 2000. Mutant torsin A, responsible for early-onset torsion dystonia, forms membrane inclusions in cultured neural cells. Human Molecular Genetics 9: 1403-1413).

A further embodiment of the present invention is related to a nanoparticle. The polynucleotides and the polypeptides of the present invention may be incorporated into the nanoparticle. The nanoparticle within the scope of the invention is meant to include particles at the single molecule level as well as those aggregates of particles that exhibit microscopic properties. Methods of using and making the above-mentioned nanoparticle can be found in the art (U.S. Pat. Nos. 6,395,253, 6,387,329, 6,383,500, 6,361,944, 6,350,515, 6,333,051, 6,323,989, 6,316,029, 6,312,731, 6,306,610, 6,288,040, 6,272,262, 6,268,222, 6,265,546, 6,262,129, 6,262,032, 6,248,724, 6,217,912, 6,217,901, 6,217,864, 6,214,560, 6,187,559,6,180,415, 6,159,445,6,149,868, 6,121,005,6,086,881, 6,007,845, 6,002,817, 5,985,353, 5,981,467, 5,962,566, 5,925,564, 5,904,936, 5,856,435, 5,792,751, 5,789,375, 5,770,580, 5,756,264, 5,705,585, 5,702,727, and 5,686,113).

A further embodiment of the present invention is related to microrarrays. The polynucleotides and the polypeptides of the present invention may be incorporated into the microarrays. The microarray within the scope of the invention is meant to include particles at the single molecule level as well as those aggregates of particles that exhibit microscopic properties. Methods of using and making the above-mentioned nanoparticle can be found in the art (U.S. Pat. No. 6,004,755)

The present invention is explained in more detail with the aid of the following embodiment examples.

### EXAMPLES

Methods and Materials

Plasmid Constructs

The tor-2 cDNA was isolated from whole worm mRNA using RT-PCR with the following primers. Primer 1 (5′-AACGCGTCGACAATGAAAAAGTTCGCTGAAAAATGGTTTCTATTG 3′) (SEQ ID NO. 11) and primer 2 (5′ AAGGCCTTCACAACTCATCATTAAACTCTTTCTTCG) (SEQ ID NO. 12). Briefly, total RNA was isolated from a mixed population of C. elegans using TriReagent (Molecular Research Center) followed by mRNA isolation using the PolyATtract mRNA Isolation System III (Promega) and cDNA synthesis using the Superscript First-Strand Synthesis System for RT-PCR from Life Technologies. Confirmation of the predicted ORF (WormBase Y37A1B.13) was performed by sequencing. Mutant versions of the tor-2 cDNA were generated using PCR-mediated site-directed mutagenesis. To obtain the Δ368 mutant form of tor-2 an initial round of PCR was performed to generate an approximately 1 kb cDNA (corresponding to amino acids 1-367) using primer 1 and primer 3 (5′ GGGAAAAATTCAAGATCAAGAACTCTTTGCATG 3′) (SEQ ID NO. 13). In parallel, an approximately 200 bp fragment (corresponding to amino acids 369-412) was amplified with primer 2 and primer 4 (5′ CATGCAAAGAGTTCTTGATCTTGAATTTTTCCC) (SEQ ID NO. 14). The two fragments were then combined and amplified using primers 1 and 2 to reconstruct the complete cDNA. The ΔNDEL form of tor-2 was also generated using PCR with the following primers. Primer 5 (5′ CTAGCTAGCATGAAAAAGTTCGCTGAAAAATGG 3′) (SEQ ID NO. 15) and primer 6, which lacks DNA encoding the terminal NDEL amino acids (SEQ ID NO. 16) (5′GGGGTACCTCAAAACTCTITCTTCGAATTGAGTG 3′) (SEQ ID NO. 17) were utilized. Mutant forms of tor-2 were confirmed by sequencing. All tor-2 cDNAs were subcloned into vector pPD30.38 using the enzymes Nhe I and Kpn I (Fire, A, Harrison, S W, Dixon, D. 1990. A modular set of lacZ fusion vectors for studying gene expression in Caenorhabditis elegans. Gene 93:189-198.).

The plasmids unc-54::Q19-GFP and unc-54::Q82-GFP were provided as a generous gift from Dr. Rick Morimoto, Northwestern University (Satyal, S, Schmidt, E, Kitagaya, K, Sondheimer, N, Lindquist, S T, Kramer, J, Morimoto, R 2000). Polyglutamine aggregates alter protein folding homeostasis in Caenorhabditis elegans. Proc Natl Acad Sci USA 97:5750-5755.). C. elegans Protocols

Nematodes were maintained using standard procedures (Brenner, S. 1974. The genetics of Caenorhabditis elegans. Genetics. 77:71-94). A mixture of the plasmids encoding the polyglutatmine-GFP fusions and torsin constructs were co-injected with the rol-6 marker gene into the gonads of early-adult hermaphrodites. The injection mixtures contained pPD30.38-Q82-GFP or pPD30.38-Q19-GFP, pRF4 (the rol-6[su1006] dominant marker) using standard microinjection procedures, and either pPD30.38-tor-2 pPD30.38-Δ368 tor-2, or pPD30.38-ΔNDELtor-2 (Mello C C, Kramer J M, Stinchcomb D, Ambros V. 1991. Efficient gene transfer in C. elegans: Extrachromosomal maintenance and integration of transforming sequences. EMBO J. 10: 3959-3970 1992). For each combination of plasmid DNAs, worm lines expressing the extrachromosomal arrays were obtained. Following stable transmission of the arrays, integration into the genome was performed using gamma irradiation with 3500-4000 rads from a Cobalt 60 (Inoue, T, Thomas, J. 2000. Targets of TGF-signaling in Caenorhabditis elegans dauer formation. Develop. Biol. 217:192-204). Stable integrated lines were obtained for all constructs.

Fluorescence Microscopy

Worms were examined using a Nikon Eclipse E800 epifluorescence microscope equipped with an Endow GFP HYQ and Texas Red HYQ filter cubes (Chroma, Inc.). Images were captured with a Spot RT CCD camera (Diagnostic Instruments, Inc.). MetaMorph Software (Universal Imaging, Inc.) was used for pseuodocoloration of images, image overlays, and aggregate size quantitation. Statistical analysis of aggregate size and quantity was performed using the software Statistica

Results

Isolation of a cDNA Encoding C. elegans TOR-2 and Site-Directed Mutagenesis

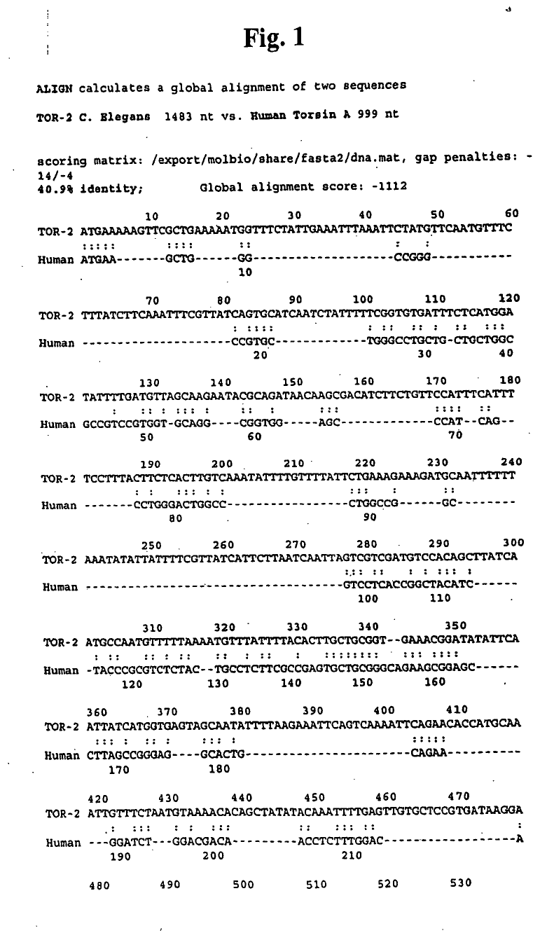

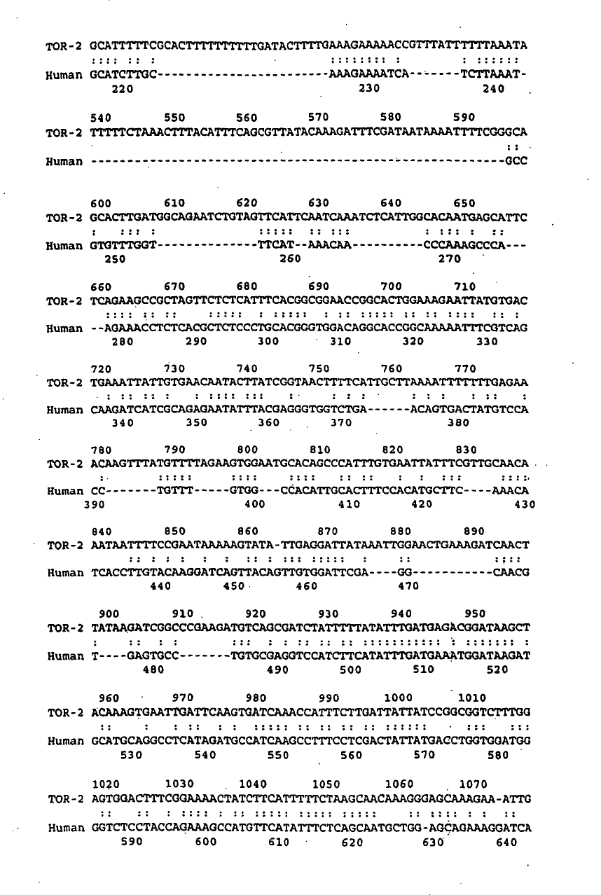

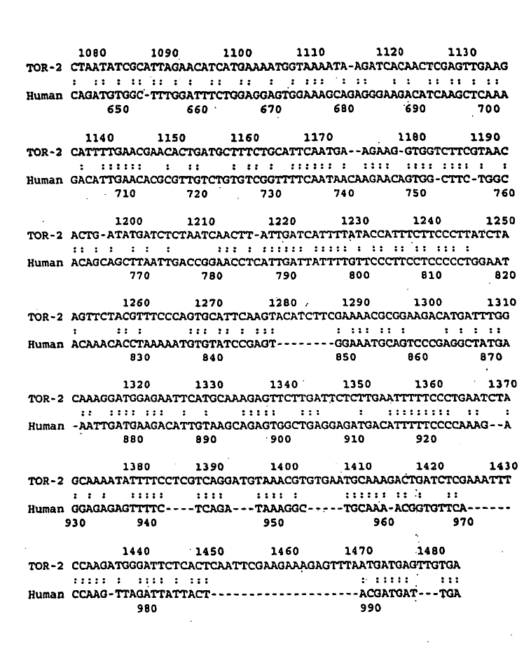

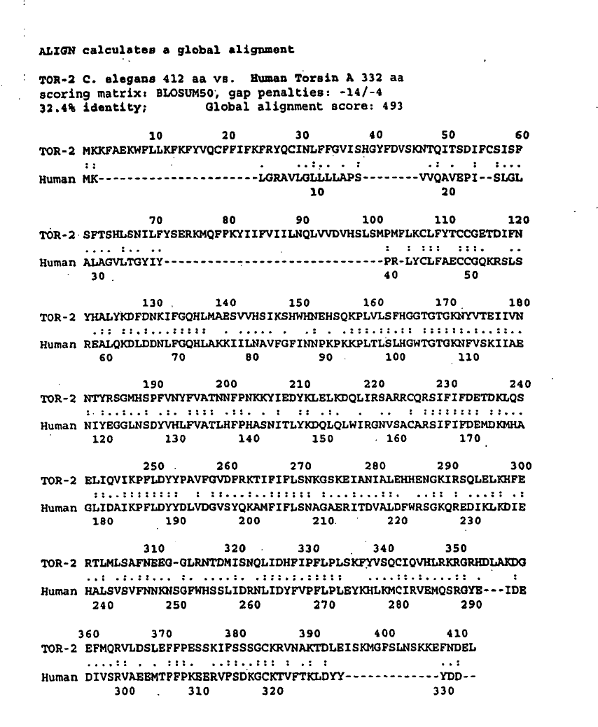

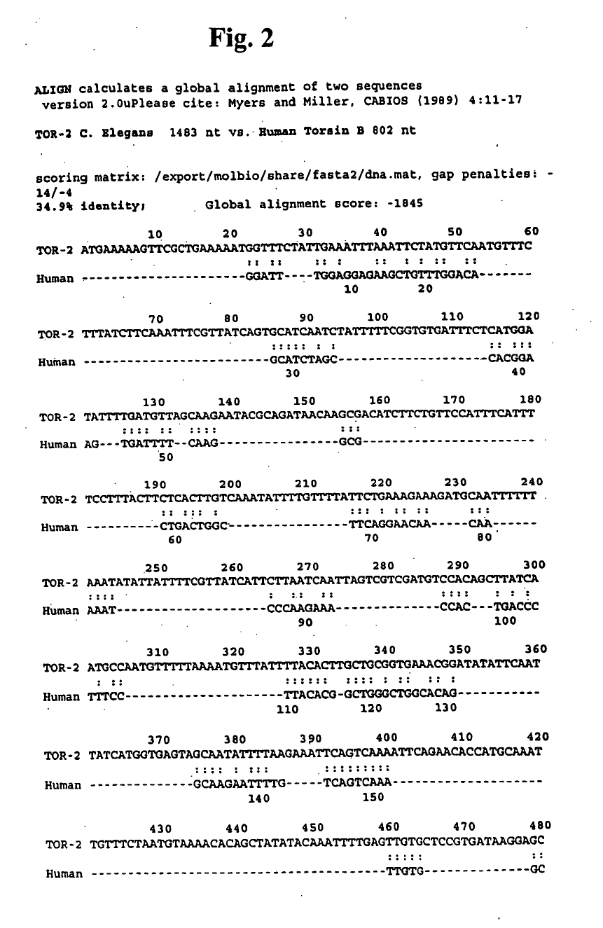

As an important resource for several lines of experimentation, a cDNA corresponding to the full-coding region predicted for the C. elegans tor-2 gene was isolated. The predicted open-reading frame was confirmed and found to be completely correct by DNA sequencing of both strands. All exon and intron boundaries were confirmed as well. This was important because the TOR-2 protein encoded by this gene contains a unique N-terminal portion not found in the other torsins of C. elegans (FIGS. 1-3). The 1.3 kb tor-2 cDNA encodes a predicted protein of 412 amino acids. A single protein from the cDNA of the approximately correct molecular weight (49 Kd) is recognized in C. elegans extracts by TOR-2 specific peptide antisera The tor-2 cDNA was subcloned into the pPD30.38 vector under the control of the C. elegans unc-54 promoter element which is expressed in body wall muscle cells (Fire, A, Harrison, S W, Dixon, D. 1990. A modular set of lacZ fusion vectors for studying gene expression in Caenorhabditis elegans. Gene 93:189-198; Satyal, S, Schmidt, E, Kitagaya, K, Sondheimer, N, Lindquist, S T, Kramer, J, Morirnoto, R. 2000). Two modifications of the tor-2 cDNA were also generated for initial structure-function analysis of the TOR-2 protein. Both of these modified cDNAs were subcloned into pPD30.38. Using site-directed mutagenesis, a cDNA designed to mimic the expression of the dominant negative protein that causes primary torsion dystonia in humans was created (Ozelius I J, Hewett J W, Page C E, Bressman S B, Kramer P L, Shalish C, de Leon D, Brin M F, Raymond D, Corey D P, Fahn S, Risch N J, Buckler A J, Gusella J F, Breakefield X O. 1997. The early-onset torsion dystonia gene (DYT1) encodes an ATP-binding protein. Nature Genetics 17: 40-48.). This consisted of a mutant tor-2 cDNA lacking a codon at amino acid 368, which encodes serine. In humans, the corresponding amino acid deletion in TOR1A is glutamic acid. Both serine and glutamic acid are polar amino acids. Additionally, a tor-2 cDNA with a deletion of the four most C-terminal amino acids (NDEL) in the TOR-2 protein was produced. The NDEL sequence is a putative ER-retention signal (data not shown).

Co-Expression of TOR-2 Suppresses Polyglutamine Repeat-Induced Protein Aggregation

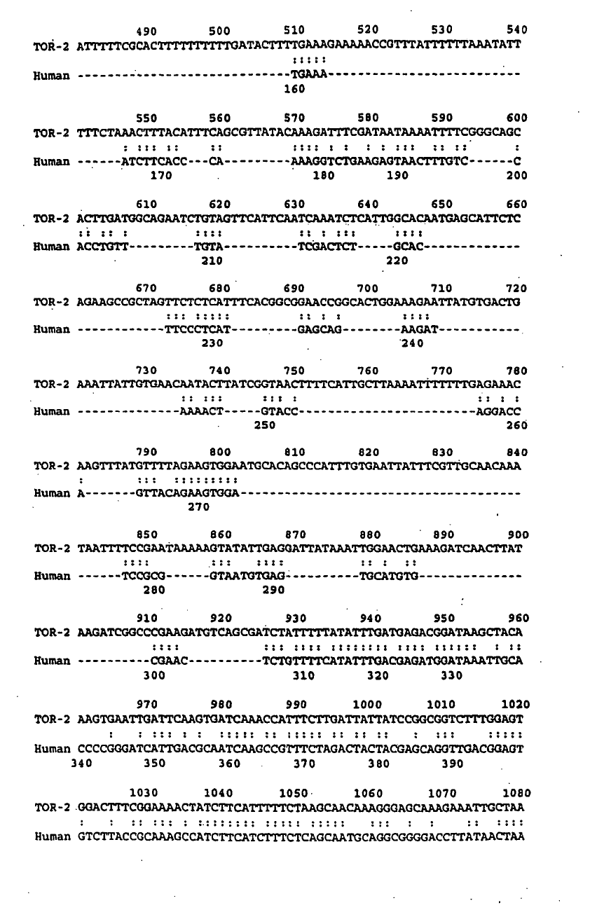

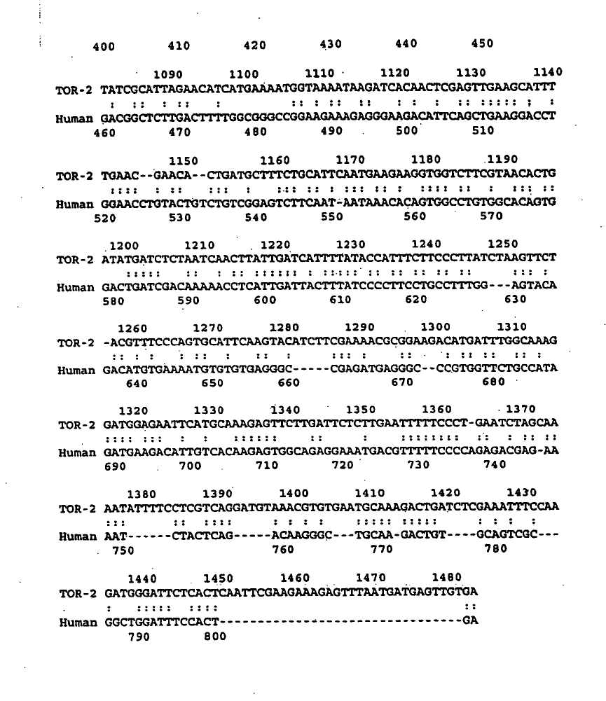

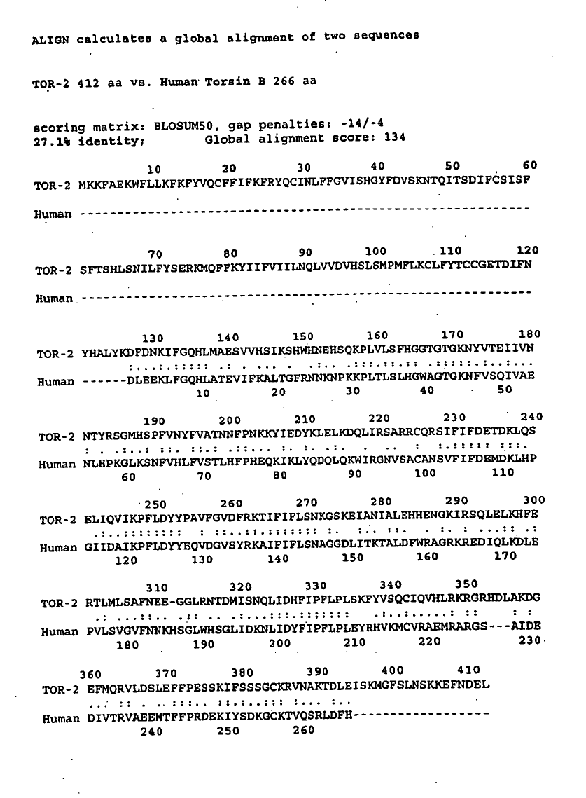

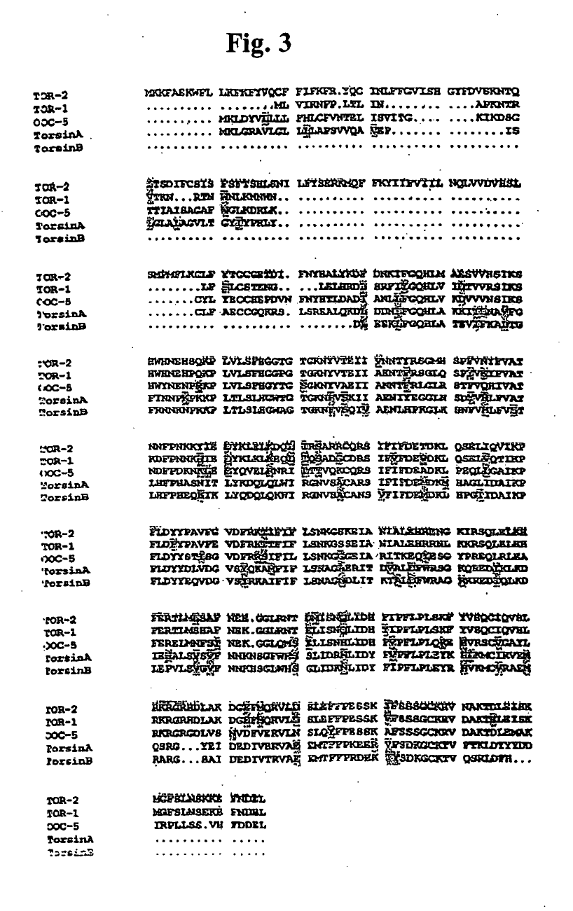

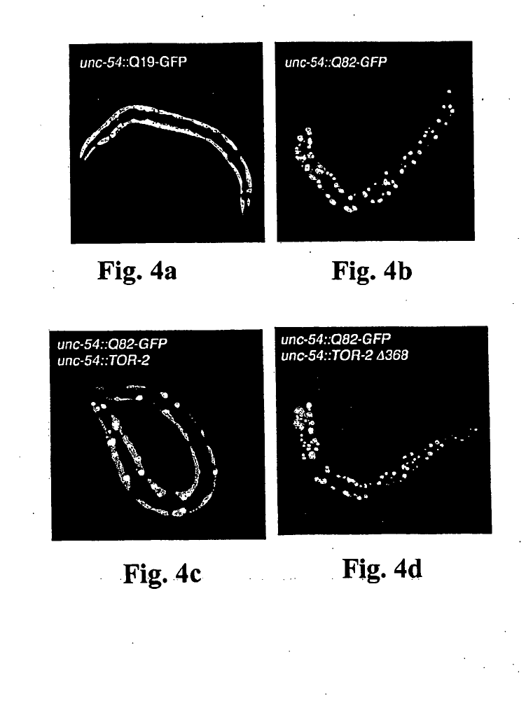

Satyal and coworkers (2000) have created artificial aggregates of polyglutamine-repeats fused to GFP that are ectopically expressed in the body wall muscle cells of C. elegans using the well characterized unc-54 promoter. Aggregation of the GFP reporter protein is dependent on the length of the polyglutamine tract. For example, body wall expression of a fusion of 19 glutamines (Q19) to GFP does not reflect a change in normally cytoplasmic, evenly distributed, and diffuse GFP localization (FIG. 4a). However, a tract of 82 glutamines (Q82) fused to GFP results in a distinct change in GFP localization wherein discrete aggregates are clearly evident in all animals (FIG. 4b).

Following introduction of the appropriate vector (unc-54::tor-2 cDNA) and selection of stable transgenic animals, co-expression of the TOR-2 protein under the control of the same high-level constitutive promoter dramatically reduces both the number of GFP-containing aggregates in animals containing Q82-GFP (FIG. 4c). In fact, diffuse body wall muscle fluorescence reappears in many of these animals as well. Co-expression of TOR-2 with Q19 does not alter the normal, cytoplasmic distribution of GFP and thus does not appear to induce aggregation. In contrast, co-expression Q82-GFP with TOR-2 containing the site-directed deletion of amino acid 368 (Δ368) in the C-terminus of this protein is not capable of restoring the body wall fluorescence in these animals (FIG. 4d). Interestingly, co-expression of TOR-2 A 368 with Q19 does not change the general cytoplasmic localization of GFP from what is found in Q19-GFP animals.

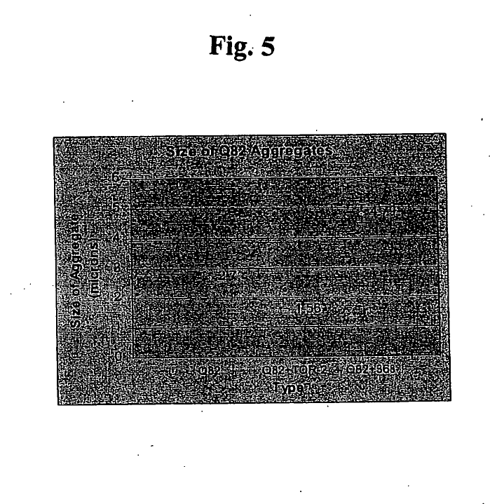

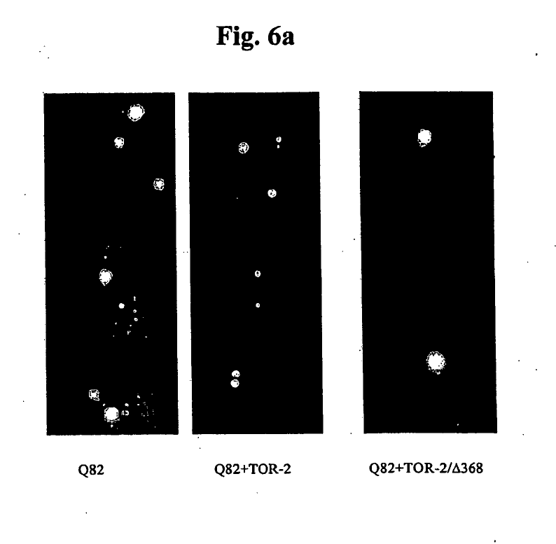

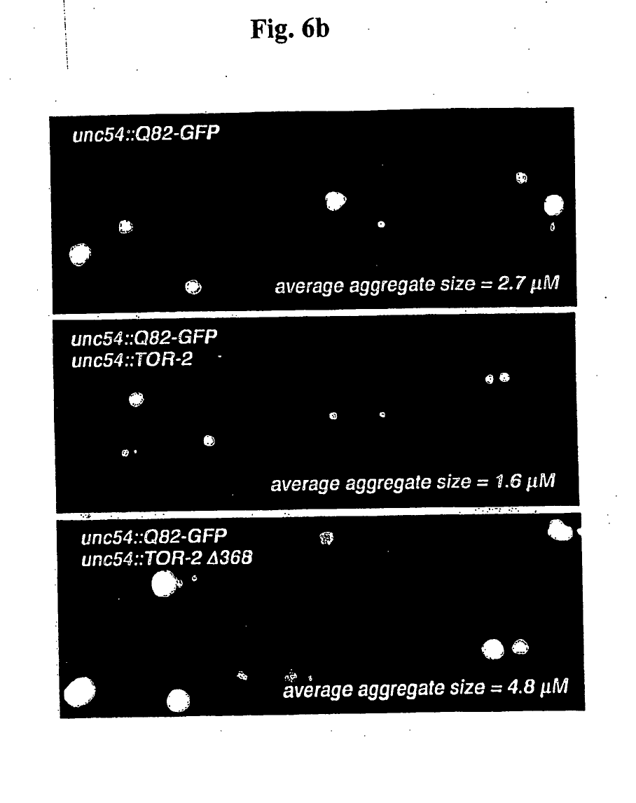

There is a statistically significant difference in the size of Q82-GFP aggregates among the various constructs. The average size of aggregates from thirty each of Q82, Q82+TOR-2, and Q82+TOR-2 Δ368 animals was recorded. The average size of aggregates from Q82 animals was 2.7 μm compared with 1.6 μm from Q82+TOR-2 (FIG. 5). This difference is significant (p<0.001) using a pair-wise t-test. Furthermore, the difference in aggregate size between Q82 and Q82+TOR-2 Δ368 animals was also significant (p<0.001) with an aggregate size of 4.8 μm for Q82+TOR-2 Δ368 animals (compared with 2.7 μm for Q82). These differences are easily observed with photomicrographs, as shown in FIG. 6a-6b.

Additionally, the amount of variability in aggregate size differs among the transgenic constructs. When aggregate size is classified into the following categories, 0-3 μm, 3-5 μm, 5-9 μm, and 9-26 μm, aggregates from Q82 animals display a 63%, 25%, 9%, and 3% distribution, respectively (Table 2). Animals co-expressing Q82 and TOR-2 demonstrate far less variability in aggregate size with 90% of the aggregates in the smallest size group and only 7% and 3% of the aggregates in the 3-5 μm and 5-9 μm categories, respectively. Conversely, the aggregates from animals co-expressing Q82 and TOR-2 Δ368 demonstrate a large degree of variability with 16% aggregates in both the 5-9 μm, and 9-26 μm categories.

TABLE 2
Variability of Q82 Aggregate Size aggregates were grouped
according to size for each different treatment. Percentages were
calculated based on the total number of aggregates for each treatment.

| Size of Aggregate | | | |
|---|---|---|---|
| (^{μ}m) | Q82 | Q82 + TOR-2 | Q82 + TOR-2/^{Δ}368 |
| 0 to 3 | 63% | 90% | 48% |
| 3 to 5 | 25% | 7% | 20% |
| 5 to 9 | 9% | 3% | 16% |
| 9 to 26 | 3% | | 16% |

There is a generalized growth defect associated with the Q82-GFP strain. This strain exhibits a reduced growth rate (as judged by larval staging at specific time points) in comparison to wild-type animals (Satyal, S, Schmidt, E, Kitagaya, K, Sondheimer, N, Lindquist, S T, Kramer, J, Morimoto, R. 2000. Polyglutamine aggregates alter protein folding homeostasis in Caenorhabditis elegans. Proc Natl Acad Sci USA 97:5750-5755). Both wild-type and mutant torsin were co-expressed with Q82-GFP in order to determine if the torsin protein alleviated this apparent homeostatic burden (FIG. 4a-4d). Co-expression with wild-type tor-2 had no obvious effect on the growth inhibition associated with Q82-GFP animals. However, tor-2 Δ368 co-expression significantly exacerbated the growth inhibitory effect such that 71% of the animals were still at the L1/L2 stage of development compared with 46% of Q82-GFP animals 48 hours after parental egg laying. Neither tor-2 Δ368 co-expression with Q19 nor wild-type tor-2 changed the growth rate of animals (See Table 3).

TABLE 3
Growth Analysis Adults were allowed to lay eggs for a set length
of time and then removed from plate. Offspring were counted
48 hours after parental removal according to larval stage.

| | L1/L2 | L3 | L4/Adult | Total |
|---|---|---|---|---|
| N2 | 2 (0.5%) | 78 (20%) | 309 (79%) | 389 |
| Q19 | 2 (14%) | 184 (63%) | 68 (23%) | 292 |
| Q82 | 134 (46%) | 149 (51%) | 7 (3%) | 290 |
| Q19/tor-2 | 99 (18%) | 395 (73%) | 46 (9%) | 540 |
| Q82/tor-2 | 122 (42%) | 140 (48%) | 27 (10%) | 289 |
| Q19/Δ368 | 44 (19.2%) | 159 (69.4%) | 26 (11.4%) | 229 |
| Q82/Δ368 | 98 (71%) | 40 (29%) | 1 (0.007%) | 139 |

Co-Expression of Other Torsin Genes Suppresses Polyglutamine Repeat-Induced Protein Aggregation.

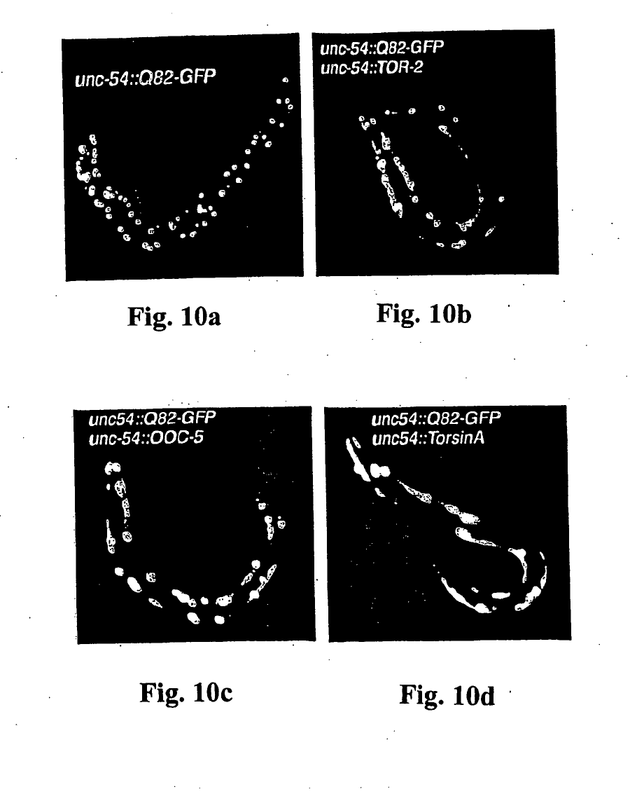

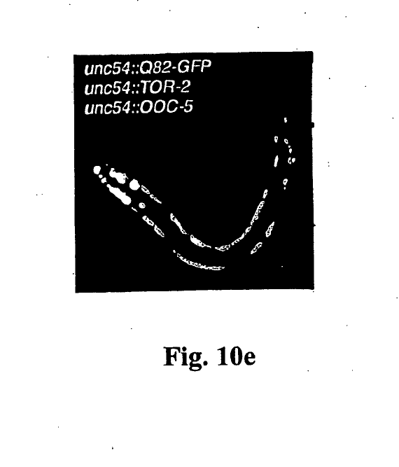

Experiments were perform in accordance with the above-described Q82+tor-2 coexpression experiments except that tor-2 was replaced with ooc-5 and TOR-A, i.e. Q82+ooc-5 and Q82+TOR-A experiments. Further, Q82 was coexpressed with ooc-5 and tor-2 (i.e. Q82+tor-2+ooc-5). FIGS. 10c-10e demonstrate that, like tor-2 alone, expression of ooc-5, TOR-A, and tor-2+ooc-5, respectively, with Q82 resulted in a more diffuse pattern of Q82 expression and a reduction of Q82 aggregates. Further, expression of TOR-2 in combination with OOC-5 results in an apparent enhanced reduction in the size of the Q82 aggregates. Perhaps, this is an indication that such torsin proteins are present at least in part in a complex.

Polyglutamine Aggregate Accumulation Over Time

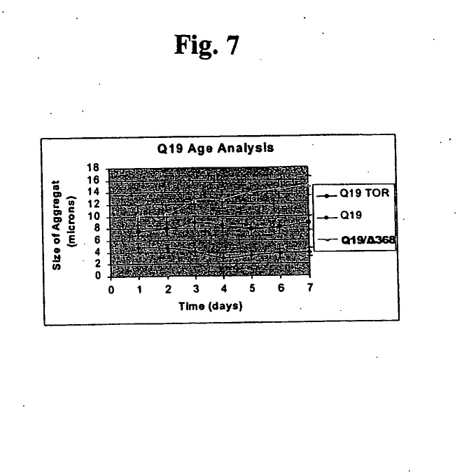

Q19-GFP animals had tiny aggregates when they reached adulthood and the aggregates increased in size as the animals aged. Specifically, adult worms expressing Q19-GFP, Q19-GFP+TOR-2, or Q19-GFP+TOR-2 Δ368 were analyzed each day for seven days and aggregate size scored (FIG. 7). Worms expressing Q19-GFP had an average aggregate size of 7.5 μm on day 1 of adulthood and 7.9 μm on day 2. The size of the aggregates increased to 8.9 μm on day 3 and decreased on day 4 to 8.5 μm. The average size fluctuated slightly on days 5, 6 and 7, but stayed close to an average size of 8.2 μm. Worms co-expressing TOR-2 were found to have significantly smaller aggregates. On day 1, the average size of the aggregates was 4.8 nm. The size of the aggregates decreased and stabilized over time with an average size of 3.0 μm on day 4 and an average size of 3.8 μm on day 6. Notably, aggregates from worms co-injected with TOR-2 Δ368 continued to increase in size each day. On the first day the average aggregate size was 10.3 μm; by day 4 it was 12.8 μm and on the last day of analysis the aggregates averaged 15.0 μm in size. Statistical analysis revealed no significant difference over time. However, there was a difference in the results of treatment and these differences persisted over time. Those with TOR-2 protein treatment had smaller aggregate size on average (3.9 μm) and were consistently smaller when compared with aggregate size for Q82, which was 8.2 μm on average. Mutant torsin protein averaged 12.8 μm and was significantly different from both wild-type torsin protein and Q82.

TOR-2 Antibody and SDS-PAGE

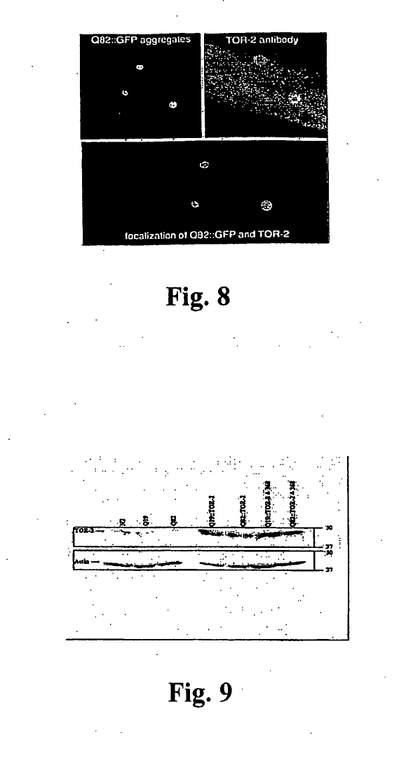

A SDS-PAGE of whole worm protein extracts and subsequent western blot were performed and the blot stained with TOR-2 antibody (FIG. 8). It showed the level of TOR-2 protein to be minimal in wild-type N2 worms, Q19 and Q82 worms. TOR-2 protein levels of Q19/TOR-2, Q82/TOR-2, Q19+TOR-2/368 and Q82+TOR-2/Δ368 revealed higher levels than N2, Q19, and Q82. However, the levels among the 4 constructs of wild-type and mutant torsin were equivalent. Actin controls were used and were determined to be equivalent for all worms used.

Antibody Staining

Whole worms stained with TOR-2 antibody showed diffuse staining throughout the worm (FIG. 9). However, distinctly higher levels of torsin localization were seen in a tight ring completely surrounding the aggregates in the Q82 worms.

Discussion

Early-onset torsion dystonia is caused by a dominant mutation resulting in the loss of a glutamic acid residue at the carboxy terminus of TOR-A. The majority of dystonia cases exhibit this deletion; this indicates that this region is critical for correct functioning of the protein. It was recently shown that members of the AAA+family form a six-member oligomeric ring. This ring structure is used in the associations with other proteins. Ozelius et al., (1997) hypothesized that this area of the glutamic acid deletion could be a critical component of the ring structure, if TOR-A forms a ring. The loss of this amino acid could affect the relationship of TOR-A with surrounding proteins (Ozelius L J, Hewett J W, Page C E, Bressman S B, Kramer P L, Shalish C, de Leon D, Brin M F, Raymond D, Corey D P, Fahn S, Risch N J, Buckler A J, Gusella J F, Breakefield X O. 1997. The early-onset torsion dystonia gene (DYT1) encodes an ATP-binding protein. Nature Genetics 17: 40-48).

An in vivo assay was utilized to examine the effects of torsins on polyglutamine aggregates. Co-expression of the TOR-2 proteins with Q82 reduced the formation of the aggregates in body-wall muscle cells. Antibody localization studies of Q82+TOR-2 revealed that the TOR-2 protein appeared to be surrounding the aggregate in a tight, doughnut-like fashion. This is interesting as it gave us the first indication of how these proteins could be interacting with the aggregates.

Formation of aggregates and their presence in intracellular inclusions is a hallmark of many neurodegenerative diseases. All cells have a system to deal with misfolded or damaged proteins. This system is called the ubiquitin-proteasome pathway (UPS). This system works by “tagging” the protein to be degraded with ubiquitin. Therefore, the protein becomes a target for degradation. However, recent reports indicate that this pathway is hindered by the presence of protein aggregates (Bence et al., 2001). By expressing two proteins known to induce the formation of aggregates, Bence et al., were able to completely restrain the UPS. This led to a buildup of proteins tagged with ubiquitin that the cells were not able to remove. This build-up, plus additional misfolded proteins, led to cell death (Bence N F, Sampat R M, Kopito R R. Impairment of the Ubiquitin-Proteasome System by Protein Aggregation. Science 292:1552-1555).

Johnston et al. (1998), described a different structure from the proteasome system called the aggresome (Johnston J A, Ward C L, Kopito R R Aggresomes: A Cellular Response to Misfolded Proteins. 1998. J of Cell Biology 143(7): 1883-1898). In a related review by Kopito et al. (2000), they describe the cell's inability to remove aggregated proteins as “cellular indigestion” (Kopito R R, Sitia R. Aggresomes and Russell Bodies. 2000. EMBO Reports 1(3): 225-231). Their theory is that aggresomes are a response to this “cellular indigestion” When the cell's ability to destroy protein aggregates is surpassed, the aggresome is formed. The formation of the aggresome is a result of cell stress. It is highly organized structurally. However, aggresomes are only formed at the microtubule organizing center (MTOC). Microtubules (MT) are used to transport the aggregated or misfolded proteins to the aggresome for degradation. Intermediate filaments are also required and are rearranged in a specific manner in order to form a supporting framework for the aggresome. Aggresomes contain high amounts of proteasomes for degradation, ubiquitin, and molecular chaperones. Interestingly, inclusions, which are found in many neurodegenerative disorders, also contain varying amounts of the same components as found in aggresomes. These inclusions contain the disease-causing protein aggregates. Therefore, there is a clear link between “cellular indigestion” and disease (Johnston J A, Ward C L, Kopito R R. Aggresomes: A Cellular Response to Misfolded Proteins. 1998. J of Cell Biology 143(7): 1883-1898; Kopito R R, Sitia R. Aggresomes and Russell Bodies. 2000. EMBO Reports 1(3): 225-231).

Based on the antibody localization and the fact that TOR-2 is able to reduce the aggregates and restore partial body-wall staining, it is interesting to speculate that perhaps TOR-2 is involved in the ubiquitin-proteasome pathway and/or in ER-associated degradation. Co-expression of the mutant tor-2, TOR-2/Δ368, with Q82 is not able to restore partial diffuse body wall staining as seen with wild-type TOR-2 and actually seemed to worsen the aggregates. This supports the theory that this portion of the gene is essential for correct functioning. Deletion of the NDEL region of tor-2, which bears homology to the ER localization signal, KDEL, did not exacerbate the aggregates as seen with the TOR-2/Δ368 (data not shown). With the deletion of the NDEL, TOR-2 is presumably not retained in the ER and is presumably free in the cytoplasm. Perhaps, it is at a higher concentration and is able to interact better with the aggregates. Also, the growth analysis data suggests that the “glutamic acid region” is critical for growth as 71% of these worms remained at L1/L2 stages 48 hours after egg-laying compared with 46% of the Q82 worms.

The data support a role for TOR-2 as a molecular chaperone. Further, the data support that TOR-A, and ooc-5 are molecular chaperones as well. This is the first clear demonstration that at least one activity of torsin proteins is chaperone activity. Further, these torsin proteins clearly reduce the amount of Q82 protein aggregation in vivo.

TOR-A is co-localized with α-synuclein in Lewy bodies of Parkinson's patients. Alpha-synuclein is misfolded in these inclusions. Torsins could help proteins fold correctly or assist in the degradation of misfolded proteins via the ubiquitin-proteasome system. The fact that the antibody localization shows the torsin protein as a tight ring around the aggregate suggests more of a degradative role. It was able to restore partial body wall staining when co-expressed with Q82, which means that the aggregates were removed. Although aggregates were still present, they were smaller when compared with Q82 alone.

The Q19 age analysis study showed that aggregates worsen over time. This is true with many diseases, such as Huntington's patients, in which the patients deteriorate as time progresses. This model could have implications for drug therapies. TOR-2 is able to reduce the aggregates. This model also showed that TOR-2 was able to keep the size of the aggregates at a baseline and stable level, while the aggregates co-expressed with TOR-2/Δ368 grew larger over time. Hopefully, TOR-2 could be used as a therapeutic agent. While it may not completely alleviate the symptoms completely, it could keep the patient's condition at a stable level instead of deteriorating as time progresses. Perhaps an enhanced effect could be observed with the co-expression of TOR-1, as these may function in a complex.

The data, combined with the aggresome theory, suggests that many diseases, such as dystonia, are the result of the cell's inability to cope with the aggregated proteins. These protein aggregates affect other proteins and could, in fact, cause a cascade-like effect. This is thought to be the mechanism behind prion diseases, such as spongioform encephalopathy. The fact that the aggregate size of TOR-2A368+Q82 is larger when compared with Q82 alone suggests that the mutant version may serve as a starting point for other proteins to misfold and form aggregates. TOR-2 appears to play a multi-dimensional role in the cell and is widely expressed.

Numerous modifications and variations on the present invention are possible in light of the above teachings. It is, therefore, to be understood that within the scope of the accompanying claims, the invention may be practiced otherwise than as specifically described herein.

All of the references, as well as their cited references, cited herein are hereby incorporated by reference with respect to relative portions related to the subject matter of the present invention and all of its embodiments.

1-61. (canceled)

62. An isolated polynucleotide, in association with a viral expression vector
wherein the polynucleotide encodes a polypeptide comprising an amino acid sequence as set forth in SEQ ID NO:8, or a fragment or a conservative substitution thereof,
and wherein the polypeptide encoded by the sequence as set forth in SEQ ID NO:8 prevents protein misfolding and aggregation.

63. The viral expression vector of claim 62, wherein the isolated polynucleotide comprises a sequence as set forth in SEQ ID NO: 7 or a fragment thereof.

64. The isolated polynucleotide of claim 62 wherein the viral vector comprises a defective retroviral, adenoviral vector or herpes-simplex viral vector.

65. An isolated host cell, comprising the vector of claim 63.

66. A method for making a torsin polypeptide, comprising culturing the host cell of claim 65 for a duration of time under conditions suitable for expression of torsin polypeptide.

67. A composition, comprising the polynucleotide of claim 62 and at least one physiologically-acceptable carrier.

68. An isolated polynucleotide, comprising a nucleic acid sequence that hybridizes to a polynucleotide comprising a sequence as set forth in SEQ ID NO:7,
wherein the isolated polynucleotide is in association with a viral vector, and
wherein the isolated polynucleotide encodes a polypeptide comprising an amino acid sequence as set forth in SEQ ID NO:8 or conservative substitutions thereof,
and wherein said polypeptide prevents protein misfolding and aggregation.

69. The isolated polynucleotide of claim 68, wherein the viral vector comprises a defective retroviral, adenoviral vector or herpes-simplex viral vector.

70. An isolated host cell, comprising the isolated polynucleotide according to claim 69.

71. A method for making a torsin polypeptide, comprising culturing the host cell according to claim 70 for a duration of time under conditions suitable for expression of torsin polypeptide.

72. A viral expression vector, comprising a nucleic acid sequence that hybridizes to a polynucleotide comprising a sequence as set forth in SEQ ID NO: 7 that encodes a polypeptide comprising an amino acid sequence as set forth in SEQ ID NO: 8, or a fragment or a conservative substitution thereof,
wherein the polypeptide prevents protein misfolding and aggregation.

73. The isolated polynucleotide of claim 68, wherein the polynucleotide hybridizes at 65° C. in the presence of a buffer comprising 0.1×SSC and 0.1% SDS to at least 15 consecutive nucleotides of SEQ ID NO:7 or at least 15 nucleotides of a complement thereof.

74. A method for producing a polynucleotide encoding a polypeptide that prevents protein misfolding and aggregation, comprising contacting a polynucleotide sample with a polynucleotide comprising at least 15 consecutive nucleotides of SEQ ID NO:7, or at least 15 consecutive nucleotides of a complement thereof.

75. A pharmaceutical composition for treating disorders associated with protein aggregation comprising an effective amount of a polynucleotide sequence hybridizing to a polynucleotide comprising a sequence as set forth in SEQ ID NO:7, wherein the polynucleotide is associated with a viral vector and at least one physiologically-acceptable carrier,
and wherein the polynucleotide sequence expresses a polypeptide comprising an amino acid sequence as set forth in SEQ ID NO:8 or a fragment or a conservative substitution thereof, wherein the polypeptide prevents protein misfolding or aggregation.

76. The pharmaceutical composition of claim 75 wherein the polynucleotide sequence comprises a sequence as set forth in SEQ ID NO:7.

77. The composition of claim 76 wherein the viral vector comprises a defective retroviral, adenoviral vector or herpes-simplex viral vector.


SEQUENCE LISTING (10 sequences)

SEQ ID NO: 1 - DNA, 1239 bp, C. elegans
atgaaaaagttcgctgaaaaatggtttctattgaaatttaaattctatgttcaatgtttctttatcttcaaatttcgttatcagtgcatcaatctatttttcggtgtgatttctcatggatattttgatgttagcaagaatacgcagataacaagcgacatcttctgttccatttcattttcctttacttctcacttgtcaaatattttgttttattctgaaagaaagatgcaattttttaaatatattattttcgttatcattcttaatcaattagtcgtcgatgtccacagcttatcaatgccaatgtttttaaaatgtttattttacacttgctgcggtgaaacggatatattcaattatcatgcgttatacaaagatttcgataataaaattttcgggcagcacttgatggcagaatctgtagttcattcaatcaaatctcattggcacaatgagcattctcagaagccgctagttctctcatttcacggcggaaccggcactggaaagaattatgtgactgaaattattgtgaacaatacttatcgaagtggaatgcacagcccatttgtgaattatttcgttgcaacaaataattttccgaataaaaagtatattgaggattataaattggaactgaaagatcaacttataagatcggcccgaagatgtcagcgatctatttttatatttgatgagacggataagctacaaagtgaattgattcaagtgatcaaaccatttcttgattattatccggcggtctttggagtggactttcggaaaactatcttcatttttctaagcaacaaagggagcaaagaaattgctaatatcgcattagaacatcatgaaaatggtaaaataagatcacaactcgagttgaagcattttgaacgaacactgatgctttctgcattcaatgaagaaggtggtcttcgtaacactgatatgatctctaatcaacttattgatcattttataccatttcttcccttatctaagttctacgtttcccagtgcattcaagtacatcttcgaaaacgcggaagacatgatttggcaaaggatggagaattcatgcaaagagttcttgattcccttgaatttttccctgaatctagcaaaatattttcctcgtcaggatgtaaacgtgtgaatgcaaagactgatctcgaaatttccaagatgggattctcactcaattcgaagaaagagtttaatgatgagttgtga

SEQ ID NO: 2 - protein, 412 aa, C. elegans
MKKFAEKWFLLKFKFYVQCFFIFKFRYQCINLFFGVISHGYFDVSKNTQITSDIFCSISFSFTSHLSNILFYSERKMQFFKYIIFVIILNQLVVDVHSLSMPMFLKCLFYTCCGETDIFNYHALYKDFDNKIFGQHLMAESVVHSIKSHWHNEHSQKPLVLSFHGGTGTGKNYVTEIIVNNTYRSGMHSPFVNYFVATNNFPNKKYIEDYKLELKDQLIRSARRCQRSIFIFDETDKLQSELIQVIKPFLDYYPAVFGVDFRKTIFIFLSNKGSKEIANIALEHHENGKIRSQLELKHFERTLMLSAFNEEGGLRNTDMISNQLIDHFIPFLPLSKFYVSQCIQVHLRKRGRHDLAKDGEFMQRVLDSLEFFPESSKIFSSSGCKRVNAKTDLEISKMGFSLNSKKEFNDEL

SEQ ID NO: 3 - DNA, 1008 bp, C. elegans
atgcttgttatcaggaattttccgcttacattgataaatgcaccaaaaaacacgcgagtcacaaaaaatagaactaattggaatcttaaaaataattggaatctcttctttctttgctctacagaaaatggactagagctacacagagatctttctagatttatttatggccagcatttggtaatagatactgtggttcgttcaatcaaatctcactggcataatgagcatcctcagaaaccgttagttctctcatttcatggtgggccgggtactggaaagaactatgtgactgaaatcatcgcaaaaaacacttttcgaagtggacttcaaagcccatatgtgaagtattttgttgcaacaaaggattttccaaacaacaaacatatagaggattataaattgaaactaaaagaacaactaatacaatctgccgacggatgtgatcgatccattttcgtgtttgatgaggtggataaattacaaagtgaactggttcaaacaatcaaaccttttcttgatttttatccagccgtttttgaagttgactttcgaaaaactaccttcatttttctaagcaacaaaggaagctctgaaattgcgaacattgcattggaacatcgtcgaaatcttaaaaaacggtcacaactcgagttgaagcattttgaacgaacacttatgtcacatgcattcaatgagaaaggtggtctacgaaacactgaattgatctctaatcagcttattgatcattatatcccatttcttcccttatctaagttctacgtttcccagtgcattcaagtacatcttcgaaaacgcggaagacatgatttggcaaaggatggagaattcatgcaaagagttctcgattctcttgaatttttccctgaatctagcaaggtattttcctcgtcaggatgtaaacgtgtagatgcaaaaactgaactagaaatttccaagatgggattctcactcaattcggagaaagagtttaatgatgagttgtag

SEQ ID NO: 4 - protein, 335 aa, C. elegans
MLVIRNFPLTLINAPKNTRVTKNRTNWNLKNNWNLFFLCSTENGLELHRDLSRFIYGQHLVIDTVVRSIKSHWHNEHPQKPLVLSFHGGPGTGKNYVTEIIAKNTFRSGLQSPYVKYFVATKDFPNNKHIEDYKLKLKEQLIQSADGCDRSIFVFDEVDKLQSELVQTIKPFLDFYPAVFEVDFRKTTFIFLSNKGSSEIANIALEHRRNLKKRSQLELKHFERTLMSHAFNEKGGLRNTELISNQLIDHYIPFLPLSKFYVSQCIQVHLRKRGRHDLAKDGEFMQRVLDSLEFFPESSKVFSSSGCKRVDAKTELEISKMGFSLNSEKEFNDEL

SEQ ID NO: 5 - DNA, 1071 bp, C. elegans
atgaaacttgattatgttcttctccttcttttccatttgtgctttgtcaacacggaattgataagcgtgattaccggtaagattaaagattccggcacgactatcgcgatatcggccggagcgttttggggtcttaaggatcgtttaaagtgttacttatatgaatgctgtcatgaacctgacgtaaatttcaattatcacacactggacgcggatattgcaaatctactgtttggccaacatttggtaaaggacgtcgttgtaaattccataaaatctcattggtacaatgaaaatccccgaaaaccactggttctttcgtttcacggttacacaggctcaggaaagaactatgtcgctgaaattatagcaaacaatacgttcaggcttggactccgcagtacttttgtccagcatatcgtggcgaccaatgattttccagacaaaaataaactggaagagtatcaagttgaattaagaaatcggattctaacaactgtacaaaaatgtcaacgatcaatattcattttcgatgaggctgataaactaccagagcagttgctaggcgcaattaaaccgttcctcgactattattcgacaatttctggcgtggattttcgaagaagcatcttcattctactaagcaataaaggtggaggagaaatagcaagaataacaaaagaacagtacgaaagtggctatccacgtgaacaactccgcttggaagctttcgaacgagaacttatgaacttctcatacaacgagaaaggtggcttacagatgagcgagctcatctcaaatcatcttatcgatcatttcgttccatttctaccgttacaacgagaacatgtcagaagttgcgttggtgcttatcttcgaaaacgaggaagaggagatctggtgtccaacgtggattttgtcgaacgagttcttaattctctacaatacttccctgaatcttccaaagcattttcttcatccgggtgtaaacgagtcgacgctaaaacggatctagaaatggccaagatacggccacttctctcaagtgttcatttcgacgatgaactataa

SEQ ID NO: 6 - protein, 356 aa, C. elegans
MKLDYVLLLLFHLCFVNTELISVITGKIKDSGTTIAISAGAFWGLKDRLKCYLYECCHEPDVNFNYHTLDADIANLLFGQHLVKDVVVNSIKSHWYNENPRKPLVLSFHGYTGSGKNYVAEIIANNTFRLGLRSTFVQHIVATNDFPDKNKLEEYQVELRNRILTTVQKCQRSIFIFDEADKLPEQLLGAIKPFLDYYSTISGVDFRRSIFILLSNKGGGEIARITKEQYESGYPREQLRLEAFERELMNFSYNEKGGLQMSELISNHLIDHFVPFLPLQREHVRSCVGAYLRKRGRGDLVSNVDFVERVLNSLQYFPESSKAFSSSGCKRVDAKTDLEMAKIRPLLSSVHFDDEL

SEQ ID NO: 7 - DNA, 999 bp, Homo sapiens
atgaagctgggccgggccgtgctgggcctgctgctgctggcgccgtccgtggtgcaggcggtggagcccatcagcctgggactggccctggccggcgtcctcaccggctacatctacccgcgtctctactgcctcttcgccgagtgctgcgggcagaagcggagccttagccgggaggcactgcagaaggatctggacgacaacctctttggacagcatcttgcaaagaaaatcatcttaaatgccgtgtttggtttcataaacaacccaaagcccaagaaacctctcacgctctccctgcacgggtggacaggcaccggcaaaaatttcgtcagcaagatcatcgcagagaatatttacgagggtggtctgaacagtgactatgtccacctgtttgtggccacattgcactttccacatgcttcaaacatcaccttgtacaaggatcagttacagttgtggattcgaggcaacgtgagtgcctgtgcgaggtccatcttcatatttgatgaaatggataagatgcatgcaggcctcatagatgccatcaagcctttcctcgactattatgacctggtggatggggtctcctaccagaaagccatgttcatatttctcagcaatgctggagcagaaaggatcacagatgtggctttggatttctggaggagtggaaagcagagggaagacatcaagctcaaagacattgaacacgcgttgtctgtgtcggttttcaataacaagaacagtggcttctggcacagcagcttaattgaccggaacctcattgattattttgttcccttcctccccctggaatacaaacacctaaaaatgtgtatccgagtggaaatgcagtcccgaggctatgaaattgatgaagacattgtaagcagagtggctgaggagatgacatttttccccaaagaggagagagttttctcagataaaggctgcaaaacggtgttcaccaagttagattattactacgatgattga

SEQ ID NO: 8 - protein, 332 aa, Homo sapiens
MKLGRAVLGLLLLAPSVVQAVEPISLGLALAGVLTGYIYPRLYCLFAECCGQKRSLSREALQKDLDDNLFGQHLAKKIILNAVFGFINNPKPKKPLTLSLHGWTGTGKNFVSKIIAENIYEGGLNSDYVHLFVATLHFPHASNITLYKDQLQLWIRGNVSACARSIFIFDEMDKMHAGLIDAIKPFLDYYDLVDGVSYQKAMFIFLSNAGAERITDVALDFWRSGKQREDIKLKDIEHALSVSVFNNKNSGFWHSSLIDRNLIDYFVPFLPLEYKHLKMCIRVEMQSRGYEIDEDIVSRVAEEMTFFPKEERVFSDKGCKTVFTKLDYYYDD

SEQ ID NO: 9 - DNA, 801 bp, Homo sapiens
gatttggaggagaagctgtttggacagcatctagccacggaagtgattttcaaggcgctgactggcttcaggaacaacaaaaatcccaagaaaccactgaccctttccttacacggctgggctggcacaggcaagaattttgtcagtcaaattgtggctgaaaatcttcacccaaaaggtctgaagagtaactttgtccacctgtttgtatcgactctgcacttccctcatgagcagaagataaaactgtaccaggaccagttacagaagtggatccgcggtaatgtgagtgcatgtgcgaactctgttttcatatttgacgagatggataaattgcaccccgggatcattgacgcaatcaagccgtttctagactactacgagcaggttgacggagtgtcttaccgcaaagccatcttcatctttctcagcaatgcaggcggggaccttataactaagacggctcttgacttttggcgggccggaagaaagagggaagacattcagctgaaggacctggaacctgtactgtctgtcggagtcttcaataataaacacagtggcctgtggcacagtggactgatcgacaaaaacctcattgattactttatccccttcctgcctttggagtacagacatgtgaaaatgtgtgtgagggccgagatgagggcccgtggttctgccatagatgaagacattgtcacaagagtggcagaggaaatgacgtttttccccagagacgagaaaatctactcagacaagggctgcaagactgtgcagtcgcggctggatttccactga

SEQ ID NO: 10 - protein, 266 aa, Homo sapiens
DLEEKLFGQHLATEVIFKALTGFRNNKNPKKPLTLSLHGWAGTGKNFVSQIVAENLHPKGLKSNFVHLFVSTLHFPHEQKIKLYQDQLQKWIRGNVSACANSVFIFDEMDKLHPGIIDAIKPFLDYYEQVDGVSYRKAIFIFLSNAGGDLITKTALDFWRAGRKREDIQLKDLEPVLSVGVFNNKHSGLWHSGLIDKNLIDYFIPFLPLEYRHVKMCVRAEMRARGSAIDEDIVTRVAEEMTFFPRDEKIYSDKGCKTVQSRLDFH